(19) United States
(12) Patent Application Publication
(10) Pub. No.: US 2009/0177122 A1
(43) Pub. Date: Jul. 9, 2009

(54) Methods for treating inflammatory skin disorders

(75) Inventor: Michael T. Peterson, Lakeville, MN (US)

Correspondence Address: ROPES & GRAY LLP, PATENT DOCKETING 39/41, ONE INTERNATIONAL PLACE, BOSTON, MA, 02110-2624, US

(73) Assignee: Celleration, Inc., Eden Prairie, MN (US)

(21) Appl. No.: 12/317,588

(22) Filed: Dec. 23, 2008

Related U.S. Application Data
(60) Provisional application No. 61/009,472, filed on Dec. 28, 2007.

(51) Int. Cl. A61N 7/00

(52) U.S. Cl. 601/2

(57) ABSTRACT

The invention provides methods for treating inflammatory skin disorders by administering low frequency ultrasound energy to decrease the inflammatory response. Exemplary skin disorders that can be treated include acne, rosacea, psoriasis, atopic dermatitis, seborrheic dermatitis, and contact dermatitis.

### RELATED APPLICATIONS

This application claims the benefit of priority to U.S. provisional application No. 61/009,472, filed Dec. 28, 2007. The specification of the foregoing application is hereby incorporated by reference in its entirety.

### BACKGROUND

Inflammatory skin disorders such as acne, rosacea, and psoriasis can have a substantial impact on the quality of life of sufferers. These conditions can be physically uncomfortable, as well as decrease self-esteem. In some cases, significant scarring may result, thereby permanently impacting appearance and self-confidence.

Current therapies for inflammatory skin disorders include antibiotics, steroids, and immunosuppressive agents. These treatments are typically delivered topically or orally. Although such therapies may provide improvement for some patients, they may also have side-effects that limit their utility. For example, long term antibiotic therapy may promote the emergence of antibiotic resistant strains of bacteria. Steroids and other immunosuppressants can place patients at increased risk for infection. Additionally, steroid therapy itself may have undesirable effects on appearance by causing weight gain, blotting, and puffiness.

Ultrasound has been used in a variety of diagnostic and therapeutic contexts. High frequency ultrasound energy has been used in diagnostic imaging and lithotripsy. Low frequency ultrasound has been used in wound debridement and to promote the healing of serious wounds. Some applications of low frequency ultrasound rely on contact between the tissue and the ultrasound transducer tip or sonotrode (See, for example, technology used by Soring and Misonix; www.soring.com; www.misonix.com). Other applications of low frequency ultrasound deliver therapeutically effective doses of energy without contact between patient tissue and the ultrasound transducer tip (See, for example, technology developed by Celleration, Inc., www.celleration.com). By avoiding contact with patient tissue, non-contact ultrasound devices and methods are particularly well suited for treating painful wound tissue.

### SUMMARY

Despite numerous uses for ultrasound energy that exist in the art, low frequency, non-contact ultrasound has not been used to provide a safe and effective treatment for inflammatory skin disorders. The present invention provides methods and devices for treating inflammatory skin disorders using low frequency ultrasound delivered without contact between the ultrasound transducer tip, or other component of the device, and the tissue to be treated. Exemplary conditions that can be treated include, but are not limited to, acne, rosacea, psoriasis, atopic dermatitis, seborrheic dermatitis, and contact dermatitis. Further exemplary conditions include, but are not limited to, boils, carbuncles, pemphigus, cellulitis, Grover's disease, hidradenitis suppurativa, and lichen planus.

The present invention is based in part on the demonstration that low frequency ultrasound energy delivered at a non-contact distance decreases expression of factors indicative of the inflammatory response. For example, low frequency, non-contact ultrasound treatment decreases both the expression of TNF-α and the activation of p38 MAPK, without affecting the viability of cells of the immune system. The ability of low frequency ultrasound to specifically modulate the inflammatory response, combined with the ease in delivering low frequency ultrasound locally to an effected area of the skin, makes it especially well suited for use in the treatment of inflammatory skin disorders. Accordingly, the present invention provides methods for treating inflammatory skin disorders.

In a first aspect, the present invention provides a method for treating an inflammatory skin disorder by delivering low frequency ultrasound energy from a non-contact distance to effected skin of a patient in need thereof. The ultrasound energy penetrates the skin to provide a therapeutic effect. Additionally or alternatively, the ultrasonic energy functions at the skin surface to provide a therapeutic effect. For example, the method results in a decrease in the inflammatory response. Over the course of one or more treatments, the use of low frequency ultrasound results in a reduction or elimination of one or more of the symptoms of the skin disorder. Additionally, over the course of one or more treatments, the use of low frequency ultrasound results in a decrease of the frequency of outbreaks of symptoms.

In certain embodiments, the inflammatory skin disorder is selected from acne, rosacea, psoriasis, atopic dermatitis, seborrheic dermatitis, and contact dermatitis. In certain other embodiments, the inflammatory skin disorders is selected from boils, carbuncles, pemphigus, cellulitis, Grover's disease, hidradenitis suppurativa, and lichen planus.

In certain embodiments, the ultrasound energy is delivered via a liquid spray, and the method comprises delivering low frequency ultrasound energy and a liquid spray from a non-contact distance to effected skin of a patient in need thereof. Exemplary liquids include, but are not limited to, saline and water. Optionally, the liquid can include a medicament such as an antibiotic, an astringent, an anti-inflammatory, a steroid, or an analgesic. In other embodiments, the liquid can include a moisturizer, skin conditioner, vitamins, or minerals. In certain embodiments, the medicament is a TNFα antagonist. In other embodiments, the liquid consists essentially of saline or water, and does not include a medicament. However, liquids that do not contain medicament can contain preservatives to improve their shelf life, or other inert agents that are not designed to have an effect on patient tissue.

In certain embodiments, the ultrasound energy is delivered without a liquid spray. In other words, the method comprises delivering ultrasound energy from a non-contact distance and in the absence of a liquid spray or coupling agent.

In certain embodiments, the ultrasound energy acts, in part, to facilitate delivery of drug to patient tissue. For example, a medicament is delivered to the ultrasound transducer to create a spray, and ultrasound energy and the spray are delivered to the patient tissue. In other embodiments, the medicament is applied topically directly to patient tissue in a first step, and ultrasound energy is then delivered from a non-contact distance to the topically applied medicament and the patient tissue. When used in this manner, ultrasound energy can be used “dry” or “wet” to facilitate penetration of both the topically applied medicament and the ultrasound energy.

In certain embodiments, the method for treating an inflammatory disorder comprises multiple treatments. For example, patients may receive doses of ultrasound two or more times per week, for one, two, three, four, or more than four weeks. Alternatively, patients may receive daily doses of ultrasound energy (daily treatments). In certain embodiments, the method comprises a single treatment.

In certain embodiments, the duration and/or frequency of treatment is varied over time depending on the severity of the patient's condition. For example, a patient who presents with severe symptoms may be initially treated daily. As the patient's symptoms decrease in severity, the frequency of treatment may be decreased to, for example, three treatments/week. As the symptoms completely subside, treatment may be discontinued entirely. Alternatively, the patient, particularly a patient at risk for recurrence of symptoms or a patient whose condition is chronic, may be placed on a prophylactic regimen of, for example, weekly treatments intended to help prevent or delay recurrence of symptoms (or, to decrease the severity of recurring symptoms).

The appropriate number of treatments, and the duration of each treatment, can be determined by a health care provider based on, for example, the particular inflammatory skin disorder being treated, the severity of the disorder, and the overall health of the patient.

In certain embodiments, each treatment comprises delivering ultrasonic energy to patient tissue for at least approximately thirty consecutive seconds. In certain embodiments, each treatment comprises delivering ultrasonic energy to patient tissue for at least approximately 1 minute, at least approximately 2 minutes, at least approximately 3 minutes, at least approximately 4 minutes, or at least approximately 5 minutes. In certain embodiments, each treatment comprises delivering ultrasonic energy to patient tissue for at least approximately 6 minutes, at least approximately 7 minutes, at least approximately 8 minutes, at least approximately 9 minutes, or at least approximately 10 minutes. In other embodiments, each treatment comprises delivering ultrasonic energy for approximately 5-10 minutes, approximately 10-15 minutes, or approximately 15-20 minutes.

In certain embodiments, the therapeutic effect includes decreasing an inflammatory response, as assayed by expression of TNF-α or other inflammatory cytokine. Therapeutic efficacy also includes one or more of decreasing bacterial count, increasing healing, and increasing proliferation of healthy skin tissue. Over the course of therapy, therapeutic efficacy can be assessed by evaluating improvement, such as a decrease in the presence, frequency, or severity of the symptoms of the inflammatory skin disorder. Exemplary symptoms include, but are not limited to, redness, itchiness, pain, fluid discharge, skin dryness, skin flaking, skin discoloration, bacterial count, swelling, size of lesions, number of lesions, frequency of lesion outbreaks, and duration of lesion outbreaks.

In certain embodiments, the low frequency ultrasound energy delivered is approximately 10-100 kHz, approximately 20-80 kHz, approximately 20-40 kHz, approximately 35-60 kHz, or approximately, 40-50 kHz. In certain embodiments, the ultrasonic energy is delivered at a frequency of approximately 20 kHz to 200 kHz. In other embodiments, the ultrasonic energy is delivered at a frequency of approximately 20 kHz to 100 kHz. In other embodiments, the ultrasonic energy is delivered at a frequency of approximately 20 kHz to 80 kHz or approximately 25 kHz to 60 kHz. In other embodiments, the ultrasonic energy is delivered at a frequency of approximately 30-50 kHz. In still other embodiments, the ultrasonic energy is delivered at a frequency of approximately 30-35 kHz, approximately 35-40 kHz, or approximately 40-45 kHz. In certain embodiments, the ultrasonic energy is delivered at a frequency of approximately 40 kHz.

In certain embodiments, the low frequency ultrasound energy is also low intensity ultrasound energy. Intensity refers to the amount of energy transferred to the tissue. In certain embodiments, the low frequency, low intensity energy has an intensity of approximately 0.1 to 2.2 W/cm². In certain embodiments, the low frequency, low intensity energy has an intensity of approximately 0.1 to 0.75 W/cm². In certain embodiments, the low frequency, low intensity energy has an intensity of approximately 0.4-0.7 W/cm². In certain embodiments, the low frequency, low intensity energy has an intensity of approximately 0.5 W/cm².

In certain embodiments, separation distance (non-contact distance) between the distal most surface of the ultrasound therapy device and the patient tissue being treated is a non-contact distance of at least 0.1 inches (2.5 mm). Preferably, the separation distance is from about 2.5 mm to about 51 cm, more preferably, from about 15 mm to about 25 mm. Regardless of the exact distance, non-contact treatment means that there is no contact between the ultrasound device and the tissue that is being treated.

In certain embodiments, the low frequency ultrasound treatment does not result in a significant increase in the temperature of the skin being treated.

In certain embodiments, the low frequency ultrasound treatment does not result in a significant decrease in viability of human cells in the treated tissue.

In certain embodiments, the low frequency ultrasound treatment decreases the symptoms of the inflammatory skin disorder and promotes healing of the effected skin without significant scarring.

In certain embodiments, low frequency ultrasound therapy is part of a therapeutic regimen used in conjunction with one or more additional treatment modalities. For example, a patient may also receive topical or oral medications, or local or systemic injections. In certain embodiments, treatment with low frequency ultrasound therapy decreases the dosage or frequency of medication used in comparison to that used in the absence of ultrasound therapy. Additionally, patients may also use diet, acupuncture, exercise, stress management, or other herbal or homeopathic therapies to help manage the symptoms of their inflammatory skin disorder.

In another aspect, the invention provides a method for reducing scarring associated with an inflammatory skin disorder. The method comprises providing a transducer which can emit low frequency ultrasonic energy. The low frequency ultrasonic energy is delivered to effected skin of said patient. The ultrasonic energy is delivered from a non-contact distance between a vibrating tip of the transducer and the effected skin of the patient, and the delivered ultrasonic energy provides a therapeutic effect to reduce scarring associated with the inflammatory skin disorder.

In certain embodiments, ultrasonic energy is delivered via a liquid spray, and the method comprises delivering the low frequency ultrasonic energy and the liquid spray to the patient. In certain embodiments, the liquid spray is generated by delivering liquid to a distal portion of the transducer. In certain embodiments, the ultrasonic energy is delivered in the absence of a liquid spray or coupling agent.

In certain embodiments, the method is part of a therapeutic regimen combining one or more additional treatment modalities. In certain embodiments, the one or more additional treatment modalities comprises applying a topical medicament to the effected skin or administering a systemic medicament prior to and/or following delivering said ultrasonic energy.

In certain embodiments, the one or more additional treatment modalities comprises a dietary regimen, an exercise regimen, yoga, heat, cold, acupuncture, acupressure, phototherapy, retinoids, oral or intravenous analgesics, anti-inflammatory agents, corticosteroids, or anti-TNFα therapeutic agents.

In certain embodiments, delivering ultrasonic energy comprises delivering ultrasonic energy for at least about 2 consecutive minutes. In other words, the methods comprises at least one treatment with ultrasonic energy, wherein the at least one treatment has a duration of at least about 2 minutes. In certain embodiments, the method comprises delivering ultrasonic energy at least twice per week for at least two weeks. In other words, the method comprises providing at least two treatments per week for at least two weeks (e.g., a total of at least four treatments).

In certain embodiments, the inflammatory skin disorder is acne. In certain embodiments, the inflammatory skin disorder is rosacea. In certain embodiments, the inflammatory skin disorder is psoriasis. In certain embodiments, the inflammatory skin disorder is atopic dermatitis. In certain embodiments, the inflammatory skin disorder is seborrheic dermatitis. In certain embodiments, the inflammatory skin disorder is contact dermatitis. In certain embodiments, the inflammatory skin disorder is boils. In certain embodiments, the inflammatory skin disorder is carbuncles. In certain embodiments, the inflammatory skin disorder is pemphigus. In certain embodiments, the inflammatory skin disorder is cellulitis. In certain embodiments, the inflammatory skin disorder is Grover's disease. In certain embodiments, the inflammatory skin disorder is hidradenitis suppurativa. In certain embodiments, the inflammatory skin disorder is lichen planus.

In another aspect, the invention provides a method for treating an inflammatory skin disorder. The method comprises providing a transducer which can emit low frequency ultrasonic energy. The ultrasonic energy is delivered to effected tissue (e.g., effected skin) from a non-contact distance between a vibrating tip of the transducer and treated patient tissue, and the ultrasonic energy provides a therapeutic effect to decrease one or more symptoms of the inflammatory skin disorder in the patient.

In certain embodiments, the ultrasonic energy acts at the skin surface to provide a therapeutic effect. In certain embodiments, the ultrasonic energy penetrates the patient tissue to provide a therapeutic effect.

In certain embodiments, the ultrasonic energy is delivered via a liquid spray. In certain embodiments, the liquid spray is generated by delivering liquid to a distal portion of the transducer. In certain embodiments, the ultrasonic energy is delivered in the absence of a liquid spray or coupling agent.

In certain embodiments, the method is part of a therapeutic regimen combining one or more additional treatment modalities. In certain embodiments, the one or more additional treatment modalities comprises applying a topical medicament to the treated tissue or systemically administering medicament prior to and/or following delivering the ultrasonic energy. In certain embodiments, the one or more additional treatment modalities comprises a dietary regimen, an exercise regimen, yoga, heat, cold, acupuncture, acupressure, phototherapy, oral or intravenous analgesics, retinoids, anti-inflammatory agents, corticosteroids, or anti-TNFα therapeutic agents.

In certain embodiments, the method comprises delivering ultrasonic energy for at least about 2 consecutive minutes. In other words, the method comprises at least one treatment and the at least one treatment has a duration of at least about 2 minutes. In certain embodiments, the method comprises delivering ultrasonic energy at least twice per week for at least two weeks. In other words, the method comprises multiple treatments occurring at least twice per week for at least two weeks.

In certain embodiments, the one or more symptoms of the inflammatory skin disorder that are treated using the ultrasonic energy are selected from one or more of expression of an inflammatory cytokine, inflammation, pain, itching, skin dryness, skin flaking, bacterial count, number of skin lesions, severity of skin lesions, frequency of outbreaks of skin lesions, redness, and skin discoloration

In certain embodiments, the inflammatory skin disorder is acne. In certain embodiments, the inflammatory skin disorder is rosacea. In certain embodiments, the inflammatory skin disorder is psoriasis. In certain embodiments, the inflammatory skin disorder is atopic dermatitis. In certain embodiments, the inflammatory skin disorder is seborrheic dermatitis. In certain embodiments, the inflammatory skin disorder is contact dermatitis. In certain embodiments, the inflammatory skin disorder is boils. In certain embodiments, the inflammatory skin disorder is carbuncles. In certain embodiments, the inflammatory skin disorder is pemphigus. In certain embodiments, the inflammatory skin disorder is cellulitis. In certain embodiments, the inflammatory skin disorder is Grover's disease. In certain embodiments, the inflammatory skin disorder is hidradenitis suppurativa. In certain embodiments, the inflammatory skin disorder is lichen planus.

In another aspect, the invention provides a method for managing symptoms of an inflammatory skin disorder. Ultrasonic energy is delivered for at least two consecutive minutes at least twice per week. In other words, ultrasonic energy treatments are administered at least twice per week. The ultrasonic energy is delivered from a non-contact distance between a vibrating tip of the transducer and treated patient tissue (e.g., skin), and the ultrasonic energy provides a therapeutic effect to treat one or more symptoms of the inflammatory skin disorder.

In another aspect, the invention provides a method for decreasing the number of skin lesions on a patient suffering from an inflammatory skin disorder. The method comprises providing a transducer which can emit low frequency ultrasonic energy. Ultrasonic energy is delivered to the patient (e.g., to effected skin of the patient). The ultrasonic energy is delivered from a non-contact distance between a vibrating tip of the transducer and treated patient tissue, and the ultrasonic energy provides a therapeutic effect to decrease the number of skin lesions on the body of the patient.

In another aspect, the invention provides a method for decreasing expression of an inflammatory cytokine in a patient having an inflammatory skin disorder. The method comprises providing a transducer which can emit low frequency ultrasonic energy. Ultrasonic energy is delivered to the patient (e.g., to effected skin of the patient), and the ultrasonic energy decreases expression of an inflammatory cytokine in effected skin tissue. In certain embodiments, the inflammatory cytokine is TNFα.

In certain embodiments, the ultrasonic energy is delivered via a liquid spray, and the method comprises delivering the low frequency ultrasonic energy and the liquid spray to the patient. In certain embodiments, liquid spray is generated by delivering liquid to a distal portion of the transducer. In certain embodiments, the ultrasonic energy is delivered in the absence of a liquid spray or coupling agent.

In certain embodiments of any of the foregoing or following aspects or embodiments, the ultrasonic energy is delivered at a frequency of approximately 200 kHz to 400 kHz. In certain embodiments of any of the foregoing or following aspects or embodiments, the ultrasonic energy is delivered at a frequency of approximately 20 kHz to 200 kHz. In certain embodiments of any of the foregoing or following aspects or embodiments, the ultrasonic energy is delivered at a frequency of approximately 30-50 kHz. In certain embodiments of any of the foregoing or following aspects or embodiments, the ultrasonic energy is delivered at a frequency of approximately 40 kHz.

In certain embodiments of any of the foregoing or following aspects or embodiments, the ultrasonic energy level provided to patient tissue is approximately 0.1-2.0 watts/cm². In certain embodiments of any of the foregoing or following aspects or embodiments, the ultrasonic energy level provided to patient tissue is approximately 0.1-1.0 watts/cm². In certain embodiments of any of the foregoing or following aspects or embodiments, the ultrasonic energy level provided to patient tissue is approximately 0.1-0.7 watts/cm².

In certain embodiments of any of the foregoing or following aspects or embodiments, the delivered ultrasonic energy acts at one or both of the skin surface and beneath the skin surface to provide a therapeutic effect. In certain embodiments of any of the foregoing or following aspects or embodiments, the delivered ultrasonic energy acts at the skin surface. In certain embodiments of any of the foregoing or following aspects or embodiments, the delivered ultrasonic energy penetrates patient tissue to a depth of at least about 1 millimeter, at least about 2 millimeters, at least about 3 millimeters, or at least about 4 millimeters. In certain embodiments of any of the foregoing or following aspects or embodiments, the delivered ultrasonic energy penetrates patient tissue to a depth of at least about 5 millimeters, at least about 6 millimeters, at least about 7 millimeters, or at least about 8 millimeters.

In certain embodiments of any of the foregoing or following aspects or embodiments, the ultrasonic energy is delivered with a liquid spray selected from a saline solution or other substantially inert liquid. In certain embodiments of any of the foregoing or following aspects or embodiments, the ultrasonic energy is delivered with a liquid spray which includes a therapeutic medicament.

For any of the foregoing methods of the present invention, including any of the aspects and embodiments described herein, the invention similarly contemplates corresponding uses of ultrasonic energy. By way of example, the invention specifically contemplates the use of low frequency ultrasonic energy delivered from a non-contact distance to decrease one or more symptoms of an inflammatory skin disorder.

In another aspect, the invention provides a method for decreasing and/or assessing expression of TNF-α in response to a stimulus. In certain embodiments, the method is an in vitro method used to modulate the expression of TNF-α in cells in vitro. In other words, cells or tissue explants cultured or maintained in vitro (outside the context of a person or whole organism) can be contacted with ultrasound energy and monitored to assess expression of TNF-α. When used in this manner, the invention provides an in vitro diagnostic method for decreasing or evaluating TNF-α expression in cells or tissue explants maintained in culture. In certain other embodiments, the method is an in vivo method. In other words, the invention provides a diagnostic method whereby TNF-α expression is monitored following in vivo treatment with ultrasound energy. Note that in vivo and in vitro refer to the status of the cells at the time the ultrasound energy is delivered. However, the assessment of TNF-α expression can occur either within or outside the context of the organism.

In certain embodiments, TNF-α expression is assessed in one or more cell types of the immune system, such as activated monocytes or macrophages. In certain embodiments, the stimulus is an allergen or irritant.

In certain embodiments, the method is used to assess the progress or effectiveness of the treatment of an inflammatory disease, such as an inflammatory skin disease. When used in this manner, assaying the expression of TNF-α or another pro-inflammatory cytokine can be used as a diagnostic to monitor improvement of the patient over the course of one or more treatments. This diagnostic step can be performed at about the same time as a therapy (just before or just after therapy). Alternatively, the diagnostic step can be performed at a different time, such as during a non-therapy day between treatments.

In another aspect, the invention provides a method for modulating the expression of one or more pro-inflammatory cytokines in response to a stimulus. In certain embodiments, the method is an in vivo method. In certain embodiments, the method is an in vitro method. In certain embodiments, the in vitro or in vivo method is used to modulate the expression of TNF-α, the activation of p38, or the expression of one or more interleukins.

In certain embodiments, expression or activation of an inflammatory cytokine is assessed in one or more cell types of the immune system, such as activated monocytes or macrophages. In certain embodiments, the stimulus is an allergen or irritant.

In another aspect, the invention provides a method of drug delivery. Ultrasound energy can be used to deliver medicament to effected patient tissue, thereby treating an inflammatory skin disorder.

In certain embodiments, the medicament is formulated for delivery as a liquid spray. When delivered as a spray, the liquid contacts the ultrasound transducer, thereby generating a liquid spray. The liquid spray and ultrasound energy are delivered to the effected tissue from a non-contact distance.

In other embodiments, the medicament is applied topically, directly to the effected tissue. Ultrasound energy is then delivered to the medicament and to the patient tissue. Without being bound by theory, the ultrasound energy facilitates the penetration of the topically applied medicament, and both the medicament and the ultrasound energy penetrate the tissue.

When ultrasound energy is used to facilitate drug delivery, medicament can be delivered more quickly and in a more targeted fashion. Additionally, given the improved tissue penetration, the use of ultrasound energy can help decrease the dosage of medicament required for therapeutic efficacy. This is particularly advantageous when administering drugs with potentially harmful side-effects, or when administering drugs that are very expensive.

The invention contemplates combinations of one or more of any of the foregoing or following aspects and embodiments of the invention.

### DETAILED DESCRIPTION OF THE DRAWINGS

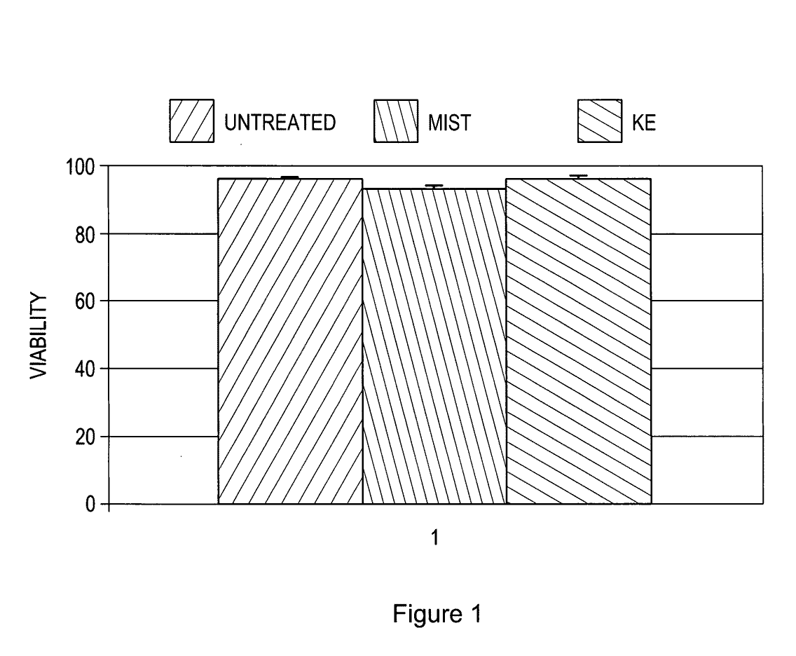

FIG. 1 shows that low frequency ultrasound energy does not significantly affect cell viability.

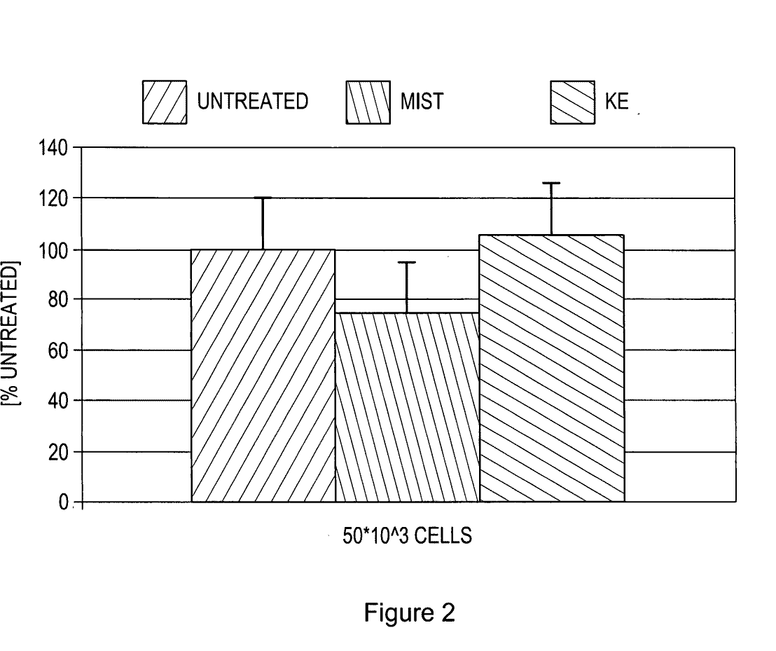

FIG. 2 shows that low frequency ultrasound energy inhibits TNFα production in LPS stimulated cells.

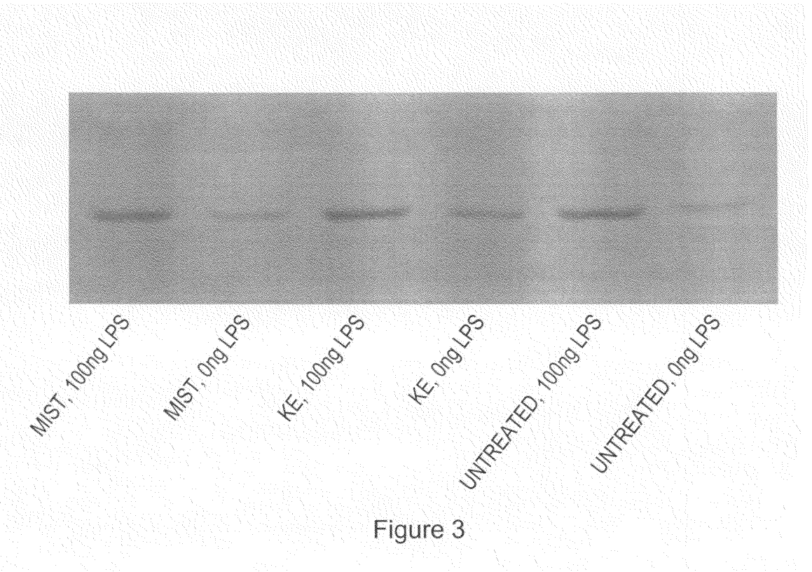

FIG. 3 shows that low frequency ultrasound energy inhibits p38 activation in LPS stimulated cells.

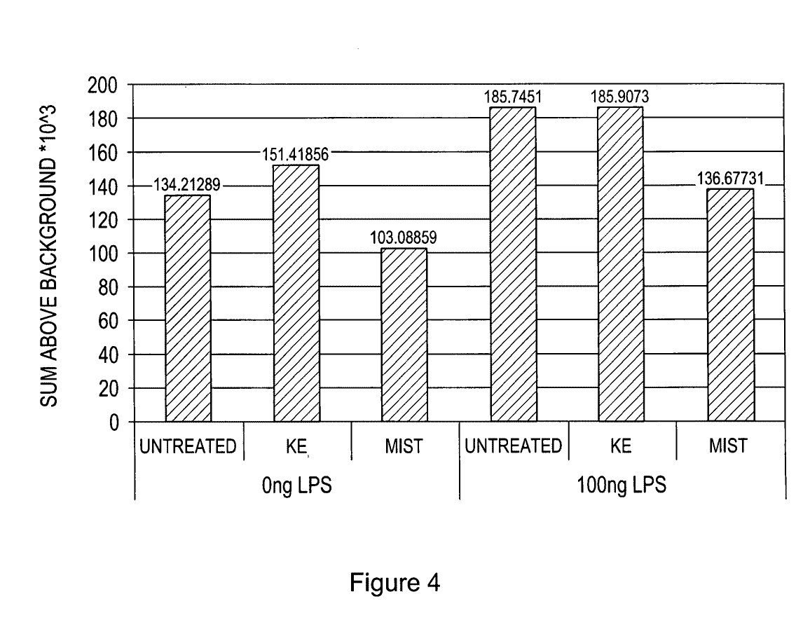

FIG. 4 shows that low frequency ultrasound energy inhibits p38 activation in LPS stimulated cells.

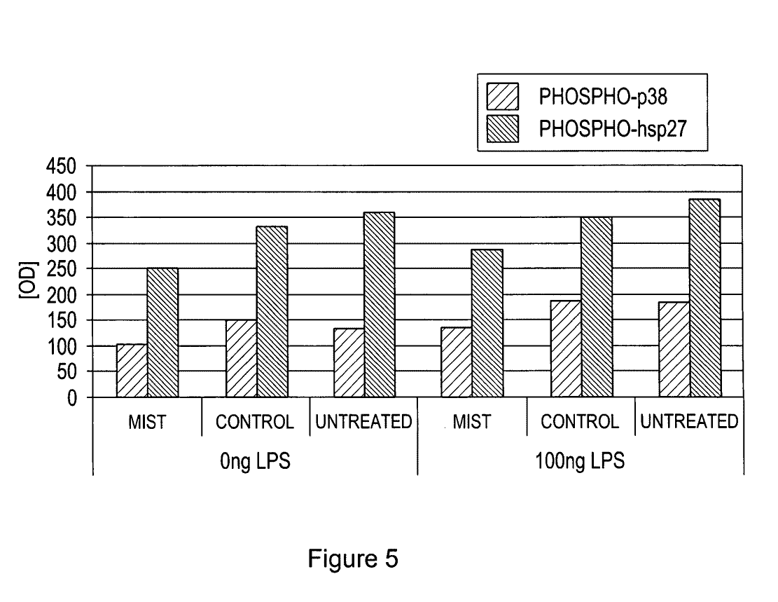

FIG. 5 shows that low frequency ultrasound energy inhibits hsp27 activation.

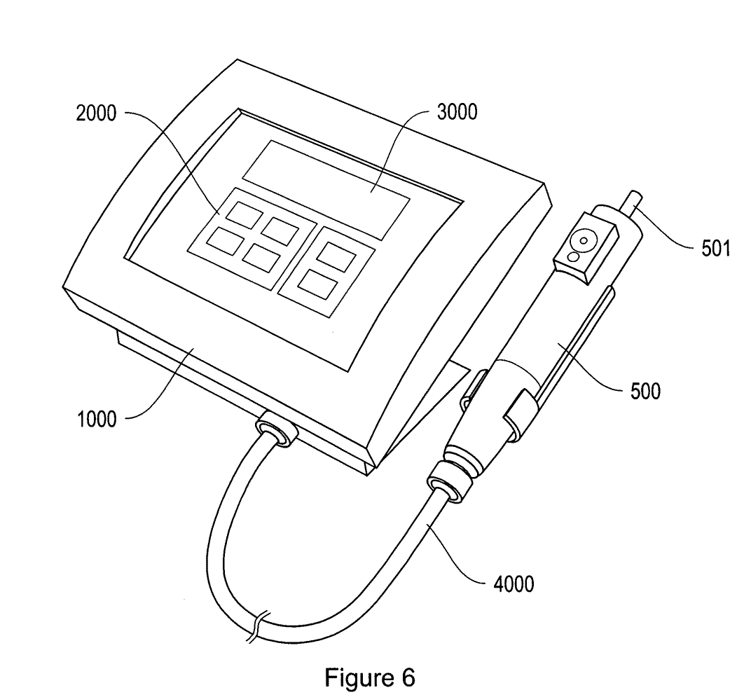

FIG. 6 shows an exemplary system for delivering ultrasonic energy to a patient.

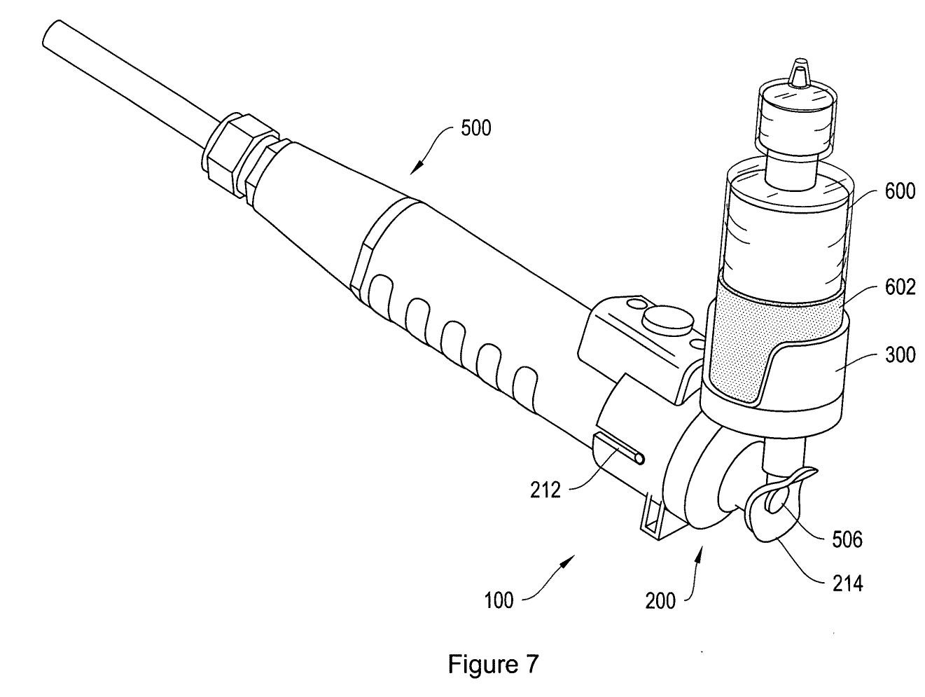

FIG. 7 shows an exemplary ultrasound transducer for delivering ultrasonic energy to a patient. The figure depicts an exemplary transducer, an applicator nozzle, and a fluid source.

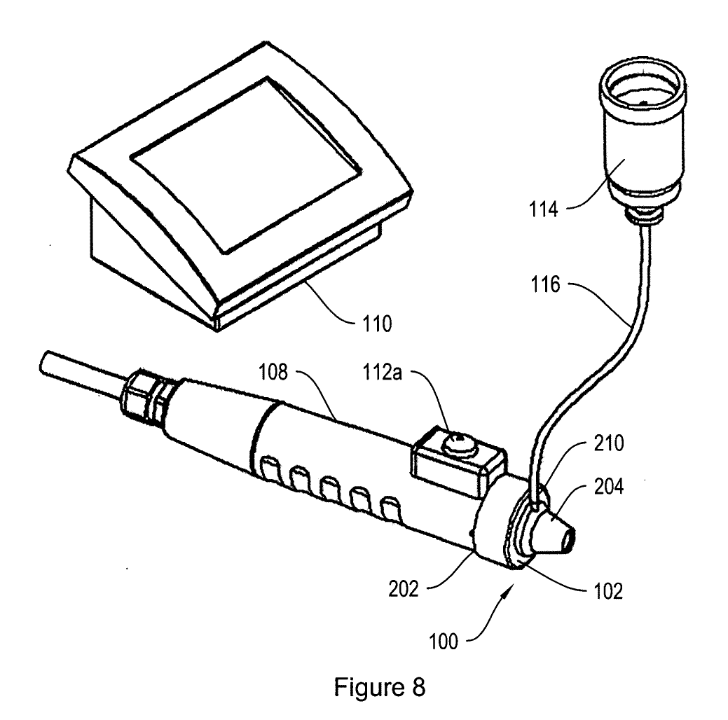

FIG. 8 shows another exemplary system for delivering ultrasonic energy to a patient. The figure depicts a system, which includes drive electronics and software for operating the device and providing information to the operator via a graphical user interface; an ultrasonic transducer; an applicator nozzle; and a fluid source.

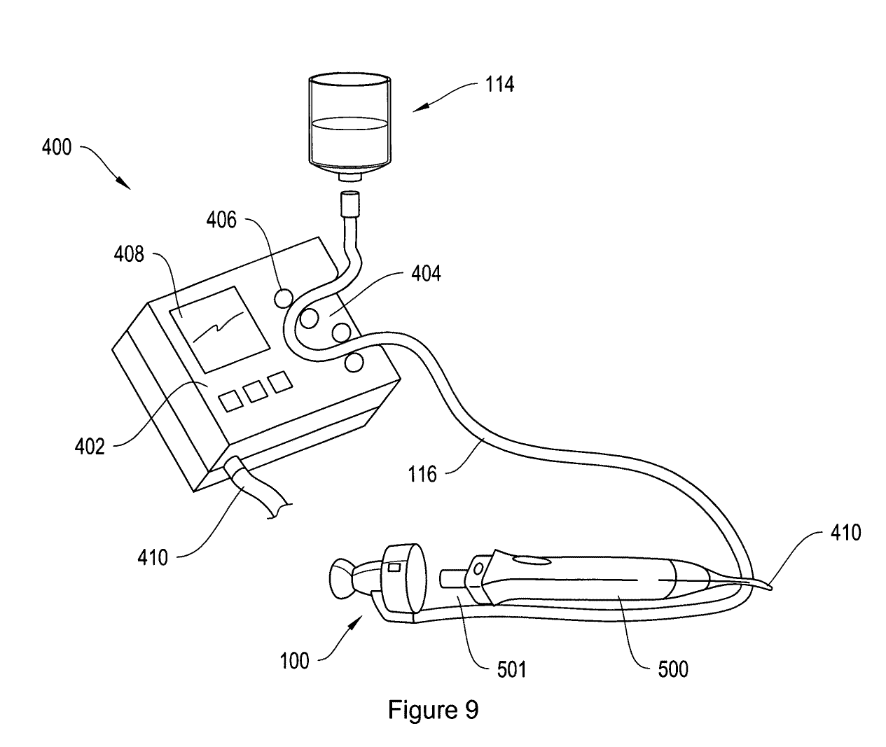

FIG. 9 shows another exemplary system for delivering ultrasonic energy to a patient. The figure depicts a system, which includes drive electronics and software for operating the device, controlling fluid flow, and providing information to the operator via a graphical user interface; an ultrasonic transducer; an applicator nozzle; and a fluid source.

### DETAILED DESCRIPTION OF THE INVENTION

Low frequency, non-contact ultrasound has been used in the treatment of wounds. U.S. Pat. No. 6,569,099, hereby incorporated by reference in its entirety, describes the use of ultrasound in wound therapy. Co-pending U.S. application Ser. Nos. 11/473,934 and 60/878,621, and 12/006,739 describe particular transducer and applicator designs, and provide further description for using non-contact ultrasound in the treatment of wounds. Co-pending application Ser. No. 11/473,934, filed Jun. 23, 2006, co-pending application Ser. No. 60/878,621, filed Jan. 4, 2007, and co-pending application Ser. No. 12/006,739, filed Jan. 4, 2008, are hereby incorporated by reference in their entirety. The systems and devices provided in these co-pending applications are exemplary of the systems and devices that can be used to deliver ultrasonic energy to patient tissue to treat an inflammatory skin disorder.

The present invention provides for the use of low frequency ultrasound, delivered at a non-contact distance, to treat or ameliorate symptoms of an inflammatory skin disorder. The methods described herein can be performed using, for example, the ultrasound therapy devices and systems disclosed in the above referenced co-pending applications. However, additional device configurations more specifically adapted for use in treating inflammatory skin disorders are also contemplated. Regardless of the specific device used, the invention provides methods for treating or ameliorating the symptoms of an inflammatory skin disorder by delivering low frequency ultrasound energy to effected tissue of a patient in need thereof. The low frequency ultrasound is delivered from a non-contact distance and without causing a substantial increase in the temperature of the treated tissue. In other words, the ultrasound energy is delivered to the effected tissue of the patient in need thereof, without contact between the ultrasound transducer, or other components of the device, and the effected tissue.

For the treatment of certain conditions, it may be preferable to have treatment conducted in a hospital or doctor's office so that a health care professional can monitor the duration and course of the treatment. Under certain circumstances, however, it may be preferable to allow the patient to be treated at home—either by a visiting health professional or by the patient himself.

The methods of the present invention can be used to treat or ameliorate one or more symptoms of an inflammatory skin disorder. The inflammatory response is an important component of the immune system. However, the inflammatory response can destroy healthy tissue and cause tissue damage. In the case of inflammatory skin disorders, patients may experience short term or long term symptoms including swelling, redness, a rash or hives, pustules, dryness, itching, and burst capillaries. Depending on the duration and severity of the symptoms, as well as the location of the lesions on the patient's body, inflammatory skin disorders can range from merely annoying to mildly embarrassing to disfiguring. Additionally, inflammatory skin disorders can be uncomfortable, or even painful. Exemplary disorders include, but are not limited to, acne, rosacea, psoriasis, atopic dermatitis, seborrheic dermatitis, or contact dermatitis. Further exemplary inflammatory skin disorders include, but are not limited to, boils, carbuncles, pemphigus, cellulitis, Grover's disease, hidradenitis suppurativa, or lichen planus. Accordingly, the invention provides methods for treating (decreasing or ameliorating one or more symptoms of) acne, rosacea, psoriasis, atopic dermatitis, seborrheic dermatitis, contact dermatitis, boils, carbuncles, pemphigus, cellulitis, Grover's disease, hidradenitis suppurativa, and lichen planus.

The present invention provides a method of treating an inflammatory skin disorder in a patient in need thereof, comprising delivering low frequency ultrasound energy from a non-contact distance to effected skin of the patient in need thereof, wherein said low frequency ultrasound energy penetrates the skin to provide a therapeutic effect to decrease symptoms of the inflammatory skin disorder.

By “treating” is meant to include decreasing or eliminating one or more symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Without being bound by theory, over the course of one or more treatments, the ultrasound energy helps decrease the local inflammatory response, thus decreasing or eliminating the presence, severity, and/or frequency of the symptoms of the inflammatory skin disorder. Regardless of the mechanism of action of the ultrasound energy, these methods can be effectively used to treat patients.

In certain embodiments, the inflammatory skin disorder is acne. In such embodiments, “treating” acne includes decreasing the severity, frequency, and/or occurrence of acne outbreaks (one or more of the symptoms of acne). In other embodiments, the inflammatory skin disorder is rosacea. In such embodiments, “treating” rosacea includes decreasing the severity, frequency, and/or occurrence of one or more of the symptoms of rosacea. In other embodiments, the inflammatory skin disorder is psoriasis. In such embodiments, “treating” psoriasis includes decreasing the severity, frequency, and/or occurrence of any one or more of the symptoms of psoriasis. In other embodiments, the inflammatory skin disorder is atopic dermatitis. In such embodiments, “treating” atopic dermatitis includes decreasing the severity, frequency, and/or occurrence of any one or more of the symptoms of atopic dermatitis. In other embodiments, the inflammatory skin disorder is seborrheic dermatitis. In such embodiments, “treating” seborrheic dermatitis includes decreasing the severity, frequency, and/or occurrence of any one or more of the symptoms of seborrheic dermatitis. In other embodiments, the inflammatory skin disorder is contact dermatitis. In such embodiments, “treating” contact dermatitis includes decreasing the severity, frequency, and/or occurrence of any one or more of the symptoms of contact dermatitis.

Exemplary symptoms include, but are not limited to, redness, swelling, pain, inflammation, itchiness, discoloration, skin dryness, skin flaking, bacterial count, cytokine expression, number of skin lesions, severity of skin lesions, and the frequency of recurrence of skin lesions.

Low frequency ultrasound energy can be delivered alone. Such methods are often referred to as delivering low frequency ultrasound “dry”. In other words, in certain embodiments, the treatment method comprises delivering low frequency ultrasound alone (from a non-contact distance) and in the absence of a liquid spray or other coupling agent. When used in this way, the ultrasound energy penetrates the tissue to provide a therapeutic effect. Over one or more treatments, improvement in the patient's condition can be observed. In certain alternative embodiments of “dry” delivery from a non-contact distance, the ultrasound energy is delivered in the absence of a liquid spray. However, prior to delivering the ultrasound energy, a coupling gel is applied to the tissue to be treated. In this embodiment, the ultrasound energy is delivered without direct contact between the ultrasound device and both the coupling gel and the patient tissue.

Alternatively, the low frequency ultrasound energy can be delivered via a liquid spray. Such methods are often referred to as delivering low frequency ultrasound “wet”. In other words, a combination of ultrasound energy and a liquid spray is delivered (from a non-contact distance) to the tissue. The energy, and to some extent the liquid spray, penetrate the tissue to provide a therapeutic effect. Exemplary liquids that can be used to generate a liquid spray include saline or water. Alternatively, the liquids used to generate the spray can themselves be (or contain) a therapeutic agent, such as an antibiotic, anti-inflammatory, steroid, analgesic, an antiseptic, and the like.

In certain embodiments, the method comprises very local delivery of ultrasound energy (in the presence or absence of a liquid spray) to the effected tissue. In other words, the goal is to treat, to the extent possible, only effected tissue and not asymptomatic tissue. In other embodiments, the method comprises local delivery that includes effected tissue, as well as adjacent tissue—even if such adjacent tissue is asymptomatic. In other embodiments, the method comprises treating the entire region. For example, if a patient has acne symptoms on the cheeks and nose, low frequency ultrasound would be delivered to the cheeks, nose, chin, forehead, and possibly even the neck. The patient's health professional can select the appropriate treatment approach, including the number of treatments, the duration of each treatment, and whether the treatment should be “dry” or “wet”.

In certain embodiments, the treated patient tissue is tissue of one or more of the head, face (e.g., cheeks, chin, forehead, nose, etc.), arms, hands, legs, or torso. In certain embodiments, the treated patient tissue is tissue of the face. In certain embodiments, the treated patient tissue is tissue of the arms or hands. In certain embodiments, the treated patient tissue is tissue of the legs. In certain embodiments, the treated patient tissue is tissue of the torso.

In certain embodiments, the method for treating an inflammatory disorder comprises multiple treatments. For example, patients may receive doses of ultrasound two or more times per week, for one, two, three, four, or more than four weeks. The appropriate number of treatments, and the duration of each treatment, can be determined by a health care provider based on, for example, the particular inflammatory skin disorder being treated, the severity of the disorder, and the overall health of the patient. In certain embodiments, each treatment comprises delivering ultrasonic energy to patient tissue for at least approximately two consecutive minutes. In certain embodiments, each treatment comprises delivering ultrasonic energy to patient tissue for at least approximately 3 minutes, at least approximately 4 minutes, or at least approximately 5 minutes. In certain embodiments, each treatment comprises delivering ultrasonic energy to patient tissue for at least approximately 6 minutes, at least approximately 7 minutes, at least approximately 8 minutes, at least approximately 9 minutes, or at least approximately 10 minutes. In other embodiments, each treatment comprises delivering ultrasonic for approximately 5-10 minutes, approximately 10-15, or approximately 15-20 minutes.

In certain embodiments, an effective amount of the delivered ultrasonic energy penetrates treated patient tissue to a depth of at least about 1 millimeter, at least about 2 millimeters, at least about 2.5 millimeters, at least about 2.75 millimeters, at least about 3 millimeters, or at least about 3.25 millimeters. In other embodiments, the delivered ultrasonic energy penetrates treated patient tissue to a depth of at least about 3.5 millimeters, at least about 3.75 millimeters, or at least about 4 millimeters. In certain embodiments, the ultrasonic energy penetrates treated patient tissue to a depth of more than about 4 millimeters (e.g., about 5, 6, 7, 8, 9, or even about 10 millimeters).

In certain embodiments, the therapeutic effect includes decreasing an inflammatory response, as assayed by expression of TNF-α or other inflammatory cytokine. Therapeutic efficacy also includes one or more of decreasing bacterial count, increasing healing, and increasing proliferation of healthy skin tissue. Over the course of therapy, therapeutic efficacy can be assessed by evaluating decrease in the presence or severity of the symptoms of the inflammatory skin disorder.

In certain embodiments, the low frequency ultrasound energy delivered is approximately 10-100 kHz, approximately 20-80 kHz, approximately 20-40 kHz, approximately 35-60 kHz, or approximately, 40-50 kHz.

In certain embodiments, the low frequency ultrasound energy is also low intensity ultrasound energy. Intensity refers to the amount of energy transferred to the tissue. In certain embodiments, the low frequency, low intensity energy has an intensity of approximately 0.1 to 2.2 W/cm². In certain embodiments, the low frequency, low intensity energy has an intensity of approximately 0.1 to 0.75 W/cm². In certain embodiments, the low frequency, low intensity energy has an intensity of approximately 0.4-0.7 W/cm². In certain embodiments, the low frequency, low intensity energy has an intensity of approximately 0.5 W/cm².

In certain embodiments, separation distance (non-contact distance) between the distal most surface of the ultrasound therapy device and the patient tissue being treated is a non-contact distance of at least 0.1 inches (2.5 mm). Preferably, the separation distance is from about 2.5 mm to about 51 cm, more preferably, from about 15 mm to about 25 mm. Regardless of the exact distance, non-contact treatment means that there is no contact between the ultrasound device and the effected tissue that is being treated. It should be noted that non-contact refers to the absence of contact with the patient tissue that is being treated. However, in certain embodiments, it is possible that components of the device may contact patient tissue that is not being subjected to treatment. For example, to facilitate delivery of the ultrasound energy, a handle of the device may be affixed to the patient's arm, thereby alleviating the need for an operator to hold the device throughout treatment. Such contact with other patient tissue that is not being subjected to treatment does not alter the characterization of the treatment as “non-contact”.

In certain embodiments, the low frequency ultrasound does not result in a significant increase in the temperature of the skin being treated.

In certain embodiments, the low frequency ultrasound decreases the symptoms of the inflammatory skin disorder and promotes healing of the effected skin tissue without significant scarring.

In certain embodiments, the low frequency ultrasound energy does not significantly decrease the viability of human cells of the effected tissue.

In certain embodiments, low frequency ultrasound therapy is part of a therapeutic regimen used in conjunction with one or more additional treatment modalities. For example, a patient may also receive topical or oral medications, or local or systemic injections. Additionally, patients can use diet, acupuncture, exercise, stress management, or other herbal or homeopathic therapies to help manage the symptoms of their inflammatory skin disorder.

In another aspect, the invention provides a method for reducing scarring associated with an inflammatory skin disorder. The method comprises providing a transducer which can emit low frequency ultrasonic energy. The low frequency ultrasonic energy is delivered to effected skin of said patient. The ultrasonic energy is delivered from a non-contact distance between a vibrating tip of the transducer and the effected skin of the patient, and the delivered ultrasonic energy provides a therapeutic effect to reduce scarring associated with the inflammatory skin disorder.

In certain embodiments, ultrasonic energy is delivered via a liquid spray, and the method comprises delivering the low frequency ultrasonic energy and the liquid spray to the patient. In certain embodiments, the liquid spray is generated by delivering liquid to a distal portion of the transducer. In certain embodiments, the ultrasonic energy is delivered in the absence of a liquid spray or coupling agent.

In certain embodiments, the method is part of a therapeutic regimen combining one or more additional treatment modalities. In certain embodiments, the one or more additional treatment modalities comprises applying a topical medicament to the effected skin or administering a systemic medicament prior to and/or following delivering said ultrasonic energy. In certain embodiments, the one or more additional treatment modalities comprises a dietary regimen, an exercise regimen, yoga, heat, cold, acupuncture, acupressure, phototherapy, retinoids, oral or intravenous analgesics, anti-inflammatory agents, corticosteroids, or anti-TNFα therapeutic agents.

In certain embodiments, delivering ultrasonic energy comprises delivering ultrasonic energy for at least about 2 consecutive minutes. In other words, the methods comprises at least one treatment with ultrasonic energy, wherein the at least one treatment has a duration of at least about 2 minutes. In certain embodiments, the method comprises delivering ultrasonic energy at least twice per week for at least two weeks. In other words, the method comprises providing at least two treatments per week for at least two weeks (e.g., a total of at least four treatments).

In certain embodiments, the inflammatory skin disorder is acne. In certain embodiments, the inflammatory skin disorder is rosacea. In certain embodiments, the inflammatory skin disorder is psoriasis. In certain embodiments, the inflammatory skin disorder is atopic dermatitis. In certain embodiments, the inflammatory skin disorder is seborrheic dermatitis. In certain embodiments, the inflammatory skin disorder is contact dermatitis. In certain embodiments, the inflammatory skin disorder is boils. In certain embodiments, the inflammatory skin disorder is carbuncles. In certain embodiments, the inflammatory skin disorder is pemphigus. In certain embodiments, the inflammatory skin disorder is cellulitis. In certain embodiments, the inflammatory skin disorder is Grover's disease. In certain embodiments, the inflammatory skin disorder is hidradenitis suppurativa. In certain embodiments, the inflammatory skin disorder is lichen planus.

In another aspect, the invention provides a method for treating an inflammatory skin disorder. The method comprises providing a transducer which can emit low frequency ultrasonic energy. The ultrasonic energy is delivered to effected tissue (e.g., effected skin) from a non-contact distance between a vibrating tip of the transducer and treated patient tissue, and the ultrasonic energy provides a therapeutic effect to decrease one or more symptoms of the inflammatory skin disorder in said patient.

In certain embodiments, the ultrasonic energy acts at the skin surface to provide a therapeutic effect. In certain embodiments, the ultrasonic energy penetrates the patient tissue to provide a therapeutic effect.

In certain embodiments, the ultrasonic energy is delivered via a liquid spray. In certain embodiments, the liquid spray is generated by delivering liquid to a distal portion of the transducer. In certain embodiments, the ultrasonic energy is delivered in the absence of a liquid spray or coupling agent.

In certain embodiments, the method is part of a therapeutic regimen combining one or more additional treatment modalities. In certain embodiments, the one or more additional treatment modalities comprises applying a topical medicament to the treated tissue or systemically administering medicament prior to and/or following delivering said ultrasonic energy. In certain embodiments, the one or more additional treatment modalities comprises a dietary regimen, an exercise regimen, yoga, heat, cold, acupuncture, acupressure, phototherapy, oral or intravenous analgesics, retinoids, anti-inflammatory agents, corticosteroids, or anti-TNFα therapeutic agents.

In certain embodiments, the method comprises delivering ultrasonic energy for at least about 2 consecutive minutes. In other words, the method comprises at least one treatment and the at least one treatment has a duration of at least about 2 minutes. In certain embodiments, the method comprises delivering ultrasonic energy at least twice per week for at least two weeks. In other words, the method comprises multiple treatments occurring at least twice per week for at least two weeks.

In certain embodiments, the one or more symptoms of the inflammatory skin disorder that are treated using the ultrasonic energy are selected from one or more of expression of an inflammatory cytokine, inflammation, pain, itching, skin dryness, skin flaking, bacterial count, number of skin lesions, severity of skin lesions, frequency of outbreaks of skin lesions, redness, and skin discoloration

In certain embodiments, the inflammatory skin disorder is acne. In certain embodiments, the inflammatory skin disorder is rosacea. In certain embodiments, the inflammatory skin disorder is psoriasis. In certain embodiments, the inflammatory skin disorder is atopic dermatitis. In certain embodiments, the inflammatory skin disorder is seborrheic dermatitis. In certain embodiments, the inflammatory skin disorder is contact dermatitis. In certain embodiments, the inflammatory skin disorder is boils. In certain embodiments, the inflammatory skin disorder is carbuncles. In certain embodiments, the inflammatory skin disorder is pemphigus. In certain embodiments, the inflammatory skin disorder is cellulitis. In certain embodiments, the inflammatory skin disorder is Grover's disease. In certain embodiments, the inflammatory skin disorder is hidradenitis suppurativa. In certain embodiments, the inflammatory skin disorder is lichen planus.

In another aspect, the invention provides a method for managing symptoms of an inflammatory skin disorder. Ultrasonic energy is delivered for at least two consecutive minutes at least twice per week. In other words, ultrasonic energy treatments are administered at least twice per week. The ultrasonic energy is delivered from a non-contact distance between a vibrating tip of the transducer and treated patient tissue (e.g., skin), and the ultrasonic energy provides a therapeutic effect to treat one or more symptoms of the inflammatory skin disorder.

In another aspect, the invention provides a method for decreasing the number of skin lesions on a patient suffering from an inflammatory skin disorder. The method comprises providing a transducer which can emit low frequency ultrasonic energy. Ultrasonic energy is delivered to the patient (e.g., to effected skin of the patient). The ultrasonic energy is delivered from a non-contact distance between a vibrating tip of the transducer and treated patient tissue, and the ultrasonic energy provides a therapeutic effect to decrease the number of skin lesions on the body of the patient.

In another aspect, the invention provides a method for decreasing expression of an inflammatory cytokine in a patient having an inflammatory skin disorder. The method comprises providing a transducer which can emit low frequency ultrasonic energy. Ultrasonic energy is delivered to the patient (e.g., to effected skin of the patient), and the ultrasonic energy decreases expression of an inflammatory cytokine in effected skin tissue. In certain embodiments, the inflammatory cytokine is TNFα.

In certain embodiments, the ultrasonic energy is delivered via a liquid spray, and the method comprises delivering the low frequency ultrasonic energy and the liquid spray to the patient. In certain embodiments, liquid spray is generated by delivering liquid to a distal portion of the transducer. In certain embodiments, the ultrasonic energy is delivered in the absence of a liquid spray or coupling agent.

In certain embodiments of any of the foregoing or following aspects or embodiments, the ultrasonic energy is delivered with a liquid spray selected from a saline solution or other substantially inert liquid. In certain embodiments of any of the foregoing or following aspects or embodiments, the ultrasonic energy is delivered with a liquid spray which includes a therapeutic medicament.

For any of the foregoing methods of the present invention, including any of the aspects and embodiments described herein, the invention similarly contemplates corresponding uses of ultrasonic energy. By way of example, the invention specifically contemplates the use of low frequency ultrasonic energy delivered from a non-contact distance to decrease one or more symptoms of an inflammatory skin disorder.

In another aspect, the invention provides a method for decreasing and/or assessing expression of TNF-α in response to a stimulus. In certain embodiments, the method is an in vitro method used to modulate the expression of TNF-α in cells in vitro. In certain embodiments, the method is an in vivo method.

In certain embodiments, TNF-α expression is assessed in one or more cell types of the immune system, such as activated monocytes or macrophages. In certain embodiments, the stimulus is an allergen or irritant.

In certain embodiments, the method is used to assess the progress or effectiveness of the treatment of an inflammatory disease, such as an inflammatory skin disease.

In another aspect, the invention provides a method for modulating the expression of one or more inflammatory cytokines in response to a stimulus. In certain embodiments, the method is an in vivo method. In certain embodiments, the method is an in vitro method. In certain embodiments, the in vitro or in vivo method is used to modulate the expression of TNF-α, the activation of p38, or the expression of one or more interleukins. Regardless of whether the energy is delivered to tissue in vivo or in vitro, cytokine expression can be evaluated using an assay performed within or outside the context of the organism.

In certain embodiments, expression or activation of an inflammatory cytokine is assessed in one or more cell types of the immune system, such as activated monocytes or macrophages. In certain embodiments, the stimulus is an allergen or irritant.

In certain embodiments of any of the foregoing or following, the delivered ultrasonic energy decreases pain. Reduction in pain can be evaluated relative to the pain experienced, on average, by patients whose treatment does not include treatment with low frequency, non-contact ultrasonic energy. Additionally or alternatively, reduction in pain may be evaluated based on the amount and frequency of pain medication requested or required to sufficiently manage patient pain relative, on average, to that needed by patient's whose treatment does not include low frequency, non-contact ultrasonic energy therapy. Such methods for evaluating reduction in pain are merely exemplary. Any standard method used by physicians and health care providers to evaluate pain and pain management can also be utilized. A reduction in reliance on pain medication includes a reduction in the dosage of medication requested or required to control pain and/or a reduction in the frequency with which medication is requested or required to adequately control pain. A reduction in reliance on pain medication may also include a shift from narcotic-based pain medications to non-narcotic or other over the counter pain medication (for example, a shift from morphine to ibuprofen).

In certain embodiments, the low frequency ultrasonic energy delivered from a non-contact distance is non-thermal. In other words, delivery of the ultrasonic energy (and optionally liquid spray) does not cause a significant increase in the temperature of the treated patient tissue (e.g., does not increase the temperature of the treated patient tissue by more than approximately 1° F.).

In certain embodiments, the ultrasonic energy is delivered via a liquid spray. Delivery of ultrasonic energy via a liquid spray is sometimes referred to herein as “wet” delivery. When used “wet”, ultrasonic energy and the liquid spray are delivered to the treated tissue from a non-contact distance (e.g., without direct contact between the device used to deliver the ultrasonic energy and the treated patient tissue). By way of example, the liquid spray can be generated by delivering liquid to a distal portion of the transducer, for example to a portion of the transducer tip.

In certain embodiments, the ultrasonic energy is delivered in the absence of a liquid spray or coupling agent. Delivery of ultrasonic energy in the absence of a liquid spray or coupling agent is sometimes referred to herein as “dry” delivery. As with “wet” delivery, the ultrasonic energy is delivered from a non-contact distance.

For any of the foregoing or following aspects and embodiments, the invention contemplates delivering low frequency ultrasonic energy. In certain embodiments, the ultrasonic energy is delivered at a frequency of approximately 200 kHz to 400 kHz. In certain embodiments, the ultrasonic energy is delivered at a frequency of approximately 20 kHz to 200 kHz. In other embodiments, the ultrasonic energy is delivered at a frequency of approximately 20 kHz to 100 kHz. In other embodiments, the ultrasonic energy is delivered at a frequency of approximately 20 kHz to 80 kHz or approximately 25 kHz to 60 kHz. In other embodiments, the ultrasonic energy is delivered at a frequency of approximately 30-50 kHz. In still other embodiments, the ultrasonic energy is delivered at a frequency of approximately 30-35 kHz, approximately 35-40 kHz, or approximately 40-45 kHz. In certain embodiments, the ultrasonic energy is delivered at a frequency of approximately 40 kHz.

For any of the foregoing or following aspects and embodiments, the invention contemplates delivering low frequency ultrasonic energy so as to provide a certain energy level to patient tissue. In certain embodiments, the ultrasonic energy level provided to patient tissue is approximately 0.1-2.0 watts/cm². In certain embodiments, the ultrasonic energy level provided to patient tissue is approximately 1.0-2.0 watts/cm². In certain embodiments, the ultrasonic energy level provided to patient tissue is approximately 0.1-1.0 watts/cm². In certain other embodiments, the ultrasonic energy level provided to patient tissue is approximately 0.1-0.7 watts/cm². In certain other embodiments, the ultrasonic energy level provided to patient tissue is approximately 0.5-1.0 watts/cm².

In certain embodiments, the methods of the present invention are repeated so that ultrasonic energy is delivered at least about twice per week for at least about two weeks. In other embodiments, ultrasonic energy is delivered at least about twice per week for at least about 3, 4, or 5 weeks. In other embodiments, ultrasonic energy is delivered at least about three times per week for at least about 2, 3, 4, or 5 weeks. In still other embodiments, the method includes one or more daily treatments for a least about 1, 2, 3, 4, or 5 weeks. In still other embodiments, the method includes one or more treatments per week for greater than six weeks. When multiple treatments are administered, each treatment may be of the same duration or of differing durations.

In certain embodiments, of the foregoing or following aspects and embodiments, the ultrasonic energy act at one or both of the skin surface or beneath the skin surface to provide a therapeutic effect. In certain embodiments, the ultrasonic energy acts at the skin surface to provide a therapeutic effect. In certain embodiments of the foregoing or following aspects and embodiments, an effective amount of the delivered ultrasonic energy penetrates treated patient tissue to a depth of at least about 1 millimeter, at least about 2 millimeters, at least about 2.5 millimeters, at least about 2.75 millimeters, at least about 3 millimeters, or at least about 3.25 millimeters. In other embodiments, the delivered ultrasonic energy penetrates treated patient tissue to a depth of at least about 3.5 millimeters, at least about 3.75 millimeters, or at least about 4 millimeters. In certain embodiments, the ultrasonic energy penetrates treated patient tissue to a depth of more than about 4 millimeters (e.g., about 5, 6, 7, 8, 9, or even about 10 millimeters). In other embodiments, the delivered ultrasonic energy penetrates treated patient tissue to a depth of at least about 4 millimeters, at least about 5 millimeters, at least about 6 millimeters, at least about 8 millimeters, at least about 9 millimeters, or at least about 10 millimeters. In other embodiments, the delivered ultrasonic energy penetrates treated patient tissue to a depth of greater than 10 millimeters. Without being bound by theory, one of skill in the art will appreciate that when all or a portion of the therapeutic benefit of ultrasonic energy is due to penetration of the energy below the tissue surface, that this indicates that an effective amount of energy penetrates to an effective depth. However, such an effective amount need not be and is likely not the same energy level as that which initially contacts the tissue surface.

Without being bound by theory, in certain embodiments, and regardless of whether and to what depth the emitted ultrasonic energy penetrates patient tissue, the emitted energy may provide a therapeutic effect at the tissue surface, and/or via a relay mechanism from the tissue surface to underlying tissue, and/or by penetrating treated tissue.

In certain embodiments of any of the foregoing, ultrasound energy is delivered from a non-contact distance “dry”. In certain embodiments of any of the foregoing, ultrasound energy is delivered from a non-contact distance in the presence of a liquid mist (“wet”). The liquid mist is generated by contacting, dripping, or otherwise delivering a liquid to a portion of a vibrating ultrasound transducer, for example, a portion of the transducer tip, to create a spray. The spray and the ultrasound energy are directed to the patient tissue. When ultrasound energy is delivered “wet”, the liquid may be an inert or substantially inert liquid such as saline solution, oil, Ringer's solution, sterile water, and the like. The liquid may also be or contain a therapeutic medicament including, but not limited to, a growth factor, antibiotic, antifungal, antimicrobial, analgesic, anti-inflammatory, hypochlorous acid, or angiogenesis promoting agent. In certain embodiments, the fluid spray produced has a substantially uniform particle size. Exemplary fluids include, but are not limited to, sterile water, oxygenated water, saline solution, oil, or other isotonic or hypertonic solution. In certain embodiments, the fluid does not contain a therapeutic drug (e.g., the fluid is substantially free from a drug). In certain other embodiments, the fluid further includes one or more therapeutic drugs such as antibiotics, anti-fungals, anti-virals, growth factors, analgesics, narcotics, and the like. When the fluid includes a therapeutic drug, the drug may be formulated in any of the foregoing fluids (e.g., water, saline, etc), or the drug may be formulated in another pharmaceutically acceptable carrier appropriate for the formulation of the particular drug. In certain embodiments, the fluid (whether including a therapeutic drug or free from therapeutic drug) further includes one or more preservatives appropriate for extending the shelf-life of the fluid. In one embodiment of any of the foregoing, the fluid (whether including a therapeutic drug or free from therapeutic drug) is sterile (e.g., the fluid is sterilized prior to or after it is added to the bottle).

The invention contemplates combinations of one or more of any of the foregoing or following aspects and embodiments of the invention.

### (ii) Definitions

Unless defined otherwise, all technical and scientific terms have the same meaning as is commonly understood by one of skill in the art to which this invention belongs.

The articles “a” and “an” are used herein to refer to one or to more than one (i.e., to at least one) of the grammatical object of the article. By way of example, “an element” means one element or more than one element.

By “treatment” is meant to refer to a session during which ultrasonic energy is delivered to patient tissue. Typically, a treatment is at least 1 consecutive minute in length.

The term “low frequency”, with respect to ultrasound energy, refers to frequencies less than approximately 500 kHz.

The term “non-contact” is used to refer to methods for delivering ultrasonic energy to patient tissue without direct contact between the treated patient tissue and the distal end of the ultrasound delivery device. When non-contact methods for delivering ultrasonic energy are used, the ultrasound transducer (including the transducer tip portion) does not contact (directly or via a coupling gel) the treated patient tissue. The non-contact distance can be measured as the distance between the distal most surface of the ultrasound transducer tip and the treated patient tissue or the non-contact distance can be measured as the distance between the distal most surface of an applicator nozzle and treated patient tissue. Exemplary non-contact distances are at least about 0.1 inches (2.5 mm) or from about 2.5 mm to about 51 cm or from about 15 mm to about 25 mm. However, recitation of an approximate non-contact distance does not indicate that the exact distance is maintained for the entire treatment time. More importantly, the term non-contact is used to indicate that there is no contact with the treated tissue. However, it is possible and permissible that components of the applicator or device may contact patient tissue that is not the intended target of treatment. For example, to facilitate delivery of the ultrasound energy, a handle of the device may be affixed to a patient's arm, thereby alleviating the need for an operator to hold the device throughout treatment. Such contact with other patient tissue that is not the intended target of treatment does not alter the characterization of the treatment as “non-contact”.

The term “applicator” and “applicator nozzle” are used interchangeably to refer to an optional portion of an ultrasound therapy device. When present, the nozzle shields the transducer tip and prevents inadvertent contact with the transducer tip when the device is in operation. Additionally, the applicator nozzle can be used as part of the mechanism for delivering fluid to a portion of the transducer and/or as part of the mechanism for directing the delivered ultrasonic energy and/or liquid spray to patient tissue. Exemplary applicator nozzles are depicted herein. However, other applicator nozzles, as well as transducer assembly designs that do not include an applicator nozzle can similarly be used.

The terms “ultrasonic energy” and “ultrasound energy” are used interchangeably herein.

### (iii) Systems, Devices and Methods for Delivering Ultrasonic Energy

Numerous systems and devices for delivering ultrasonic energy are available. Such existing devices, as well as modifications and improvements thereof, are exemplary of systems and devices that may be used to deliver low frequency ultrasonic energy to patient tissue. In certain embodiments, low frequency ultrasonic energy is delivered from a non-contact distance and without direct contact with treated patient tissue. For example, the low frequency ultrasonic energy (in the presence or absence of liquid spray) is delivered from a non-contact distance between the treated patient tissue and the transducer tip of the ultrasound device and/or the applicator nozzle.

FIG. 6 depicts an exemplary system for delivering ultrasonic energy. An exemplary ultrasound therapy device includes a transducer assembly 500 operatively connected via a connector 4000 to a generator 1000. As described herein, the ultrasound therapy device may further include an applicator 100 (not shown in FIG. 6) that can be interconnected to the transducer assembly 500, thereby shielding the transducer tip portion 501.

Briefly, the generator 1000 includes the components necessary to supply power to the transducer assembly 500, and also contains a control panel 2000, and a graphical user interface (GUI) 3000 for displaying information helpful to the operator. The generator 1000 includes three major functional sections: the AC MAINS, the main board, and the GUI board. The local AC MAINS is connected to an appliance inlet with a hospital grade detachable power cord. The appliance inlet is a power entry module listed for medical applications. In certain embodiments, the appliance inlet is a power entry module with an 115V/230V voltage selection, and is designed to operate on 115 Vac and 60 Hz (e.g., for operation in North America) or 230 Vac and 50 Hz (e.g., for operation in Europe).

The MAIN board converts the secondary output voltage from the MAINS transformer to the low voltage power rails for the internal electronics and the drive voltage for the drive electronics to the transducer assembly. The MAIN board contains a microprocessor that controls, measures, and monitors the drive electronics. The transducer assembly connects to the MAIN board. The microprocessor, referred to as the engine, monitors the performance of the system and communicates the information to a second microprocessor located on the GUI board. In certain embodiments, the engine communicates to the second microprocessor via a RS-232 communication link. In certain embodiments, the electronics drive the ultrasound portion of the drive electronics with a push-pull converter that has a feedback loop with a Phase Locked Loop (PLL) to track the center frequency of the ultrasound components.

The GUI board provides the graphical user interface for the operator. A custom membrane switch panel with, for example 6 keys, allows the operator to select the functions and operating parameters of the system. A purchased graphical LCD display, connected to the GUI board, can be used to display information to the operator. For example, information about the system's status, mode of operation, and treatment time can be displayed via the GUI. The GUI may have a back light generator for the LCD on it. The GUI microprocessor runs the system by controlling the human interface and running various algorithms to control the operation of the system. For example, a treatment algorithm can be run on the GUI microprocessor. In certain embodiments, the system may include one or more of a timer to record total treatment time, a timer to count-down from a selected treatment time to zero, and an alarm to indicate that the total treatment time has elapsed or that there is a problem with some component of the device.

FIG. 6 also depicts an example of a transducer assembly 500. As depicted, only the transducer tip portion 501 is visible. The remainder of the transducer is contained within the plastic casing of the assembly. As depicted, the transducer tip portion 501 is uncovered. In operation, the transducer tip portion 501 may be shielded with, for example, an applicator nozzle. Exemplary applicator nozzles 100 are depicted in FIGS. 7-9. When used, an applicator nozzle helps prevent inadvertent contact of either the operator or the patient with the vibrating tip portion of the transducer. Additionally, an applicator nozzle can be used as part of the mechanism for directing the delivered ultrasonic energy to patient tissue. When the system is used “wet”, the applicator nozzle can also be used to deliver fluid to the transducer tip portion and to direct the delivered ultrasonic energy and the fluid spray to patient tissue.

The system depicted in FIG. 6 is currently sold by Celleration, Inc. as part of the MIST Therapy® System.

FIG. 7 shows an example of a portion of a system for delivering ultrasonic energy. Specifically, FIG. 7 shows a transducer assembly 500 interconnected to one embodiment of an applicator nozzle 100. The transducer assembly can be operatively interconnected to a generator, for example generator 1000 shown in FIG. 6.

As depicted, the transducer assembly 500 and applicator nozzle 100 are ready to be used “wet”. Specifically, a fluid bottle 600 containing fluid 602 is interfitted to a portion of the applicator nozzle so that fluid can be delivered to the transducer tip portion, and so that ultrasonic energy and a fluid spray can be delivered to patient tissue. As depicted, a fluid bottle 600 is interfitted to a cup portion 300 of the applicator nozzle. As shown, fluid delivery to the transducer would largely be gravity driven. However, pressure delivery methods, peristaltic pumps, fluid cartridges affixed directly to or housed within the transducer assembly, and the like are similarly contemplated. An alternative mechanism for providing fluid to the transducer is via a sock, membrane, film, or other means to wick fluid from a fluid container or fluid line to all or a portion of the transducer.

In certain embodiments, an applicator, as described herein, is interconnected with an ultrasound therapy device and used to deliver ultrasound energy (in the presence or absence of a liquid spray) to patient tissue. When used in this manner, the ultrasound energy (and liquid spray, if present) is delivered without contact between the applicator and the patient tissue being treated. In other words, the ultrasound energy (and liquid spray, if present) are delivered from a non-contact distance. Once delivered, the ultrasound energy acts at the cell surface and/or penetrates the treated tissue to provide a therapeutic effect.

The transducer assembly 500 and applicator nozzle 100 depicted in FIG. 7 are currently sold by Celleration, Inc. as part of the MIST Therapy® System. As depicted, applicator 100 generally includes a nozzle 200 and a cup 300. However, applicator designs that exclude the cup 300 can be readily used.

When included in the applicator design, the cup 300 may be designed to hold at least a portion of a bottle 600 therein. The bottle 600 generally holds a fluid 602, which may be saline. The fluid may alternatively be sterile water or some other isotonic or hypertonic solution or combination of solutions. The fluid may consist entirely or essentially of the saline or other similar solution, or the fluid may optionally include a therapeutic drug. The fluid may optionally be sterilized.

The applicator 100 is mechanically connectable with a transducer assembly 500 of an ultrasound therapy device. When activated, the transducer assembly 500 produces ultrasonic waves having a frequency and capable of delivering ultrasonic energy to patient tissue.

The proximal portion of the nozzle 200 slides over a distal portion of the transducer assembly 500. The plurality of aligning slots 212 (illustrated as two slots) of the nozzle 200 engage with a plurality of aligning pins of the transducer assembly 500. When connected, the distal end 506 of a transducer tip portion of the transducer assembly 500 may extend distally of the distal opening 214 of the nozzle 200 but not to a location that is distal of the tip 205 of the nozzle 200.

The fluid 602 to be sprayed and provided within the bottle 600 (or other appropriate fluid container or vessel) can be any appropriate carrier, such as saline, water (regular or distilled), or oil to be applied to tissue, such as a vegetable, peanut, or canola oil, optionally with a soluble pharmaceutical (e.g., an antibiotic), antiseptic, conditioner, surfactant, emollient, or other active ingredient. The fluid 602 can also be a combination of two or more fluids and/or substances having microscopic particles, such as powder and the like. Exemplary fluids include, but are not limited to, sterile water, saline solution, oil, oxygenated water, or other isotonic or hypertonic solutions. Exemplary fluids may, in certain embodiments, further include drugs (e.g., therapeutic agents) such as antibiotics, anti-fungals, anti-virals, growth factors, analgesics, narcotics, and the like, formulated in any of the foregoing fluids or in other pharmaceutically acceptable fluids appropriate for the formulation of the particular drug. However, in certain embodiments, the fluid does not include a drug. The fluid may be sterilized so that, in use, a spray of a sterile solution is administered to patients.

It is envisioned for the bottle 600 of the present disclosure to be eliminated and/or replaced with another structure for delivering the fluid 602 to the transducer assembly 500, such as a fluid bag or integrated cartridge or canister (not shown). In such an embodiment, the fluid 602 may optionally be delivered to the transducer assembly 500 in a pressurized state. Desirably, the pressurized fluid 602 in such an embodiment may be approximately equal to the pressure of the fluid 602 exiting the bottle 600, as in the previous embodiment.

FIG. 8 shows another example of a portion of a system for delivering ultrasonic energy. Specifically, FIG. 8 shows a generator 1000, a transducer assembly 500, and an alternative design for an applicator nozzle 100.

FIG. 8 depicts an applicator 100. As depicted the applicator 100 is interconnected to a transducer assembly 500. The applicator 100 is also interconnected to a fluid source 114 via a flexible tubing 116.

FIG. 8 also shows a switch 112a that may control one or more of the power supplied to the transducer assembly 500, the flow of fluid, or the fluid flow rate. Also shown is a fluid source 114 and tubing 116 that interconnects the fluid source 114 to the applicator 100 via a connector 210. As depicted, the connector comprises an opening in communication with the interior of the applicator 100, thereby providing a conduit to deliver fluid to a portion of the transducer.

FIG. 9 shows another example of a portion of a system for delivering ultrasonic energy. Specifically, FIG. 9 shows a pump-generator 400, a transducer assembly 500, and an alternative design for an applicator nozzle 100.

As depicted the transducer assembly and applicator are interconnected to a fluid source 114 via flexible tubing 116. The applicator 100 is depicted just prior to interconnection to the transducer assembly 500. The transducer tip portion 501 is visible. When present, and once the applicator 100 is interfitted to the transducer assembly 500, the transducer tip portion 501 will be shielded, thereby preventing inadvertent contact with the transducer tip portion 501.

In this depiction, the pump-generator 400 includes additional mechanisms for controlling fluid delivery to the transducer assembly 500, the transducer tip portion 501, and the applicator 100. The depicted system provides an example of a fluid delivery mechanism that is not gravity fed, but rather under direct control of the user. The use of a peristaltic pump, such as the pump depicted, permits additional control over the rate at which fluid is delivered to the transducer.

An exemplary peristaltic pump at least includes a rotor and rollers or other tube-engaging members movable within a housing relative to the clamped flexible tubing. A peristaltic pump typically includes between four to six rollers. The rollers compress the clamped flexible tubing. As the rotor turns, the part of the tube under compression gets pinched and the pinching motion forces the fluid to move through the tube. The rollers relax the clamped flexible tubing as the rotor turns and the flexible tubing opens to its original state to induce fluid flow. FIG. 9 shows a fluid container 114, a tubing 116, an applicator 100, and a generator-pump unit 400. The generator-pump unit 400 includes, among other things, a generator portion 402, a pump portion 404, multiple rollers 406, an LCD display 408, and a connection inlet 410. The generator portion 402 may automate the fluid to enter the nozzle by, for example, regulating a valve (not shown) coupled to the tubing 116. In addition, the pressure applied to the fluid may be automatically maintained by the generator 402 based on values supplied by the user from a user interface, such as a dial, coupled to the generator 402. In addition, the generator 402 may report to the user the monitored pressure readings in the LCD display 404 of the generator 402. Although not shown, the generator-pump unit 400 may include an outer cover to protect the rollers 406 and the flexible tubing. In certain embodiments, the generator-pump unit 400 is fully integrated such that it performs all of the functions of the generator 1000 depicted in FIG. 6.

Although not depicted in the foregoing figures, fluid flows into the nozzle and is delivered to a vibrating transducer tip portion 501. Fluid delivery can be, for example, gravity driven or mechanically or otherwise controlled. The fluid source can be separate from or integrated within the generator and/or transducer assembly. Fluid delivery can be along all or a portion of the transducer tip portion, including to a distal portion of the tip portion. Fluid is dripped, flowed, wicked, or otherwise applied to all or a portion of the transducer tip portion, including to a plurality of sections of the transducer tip portion. Ultimately, in operation, fluid is delivered from the distal radiation surface of the transducer tip portion and ultrasonic energy and a fluid spray is delivered to patient tissue. Fluid contacts the transducer tip portion and ultrasonic energy and a fluid spray are delivered from the distal end of the applicator nozzle.

FIGS. 6-9 are merely exemplary of systems and devices that can be used to deliver ultrasonic energy. Additionally, although not depicted, devices that are typically used to deliver ultrasonic energy via direct contact with patient tissue can be adapted for use at a non-contact distance as part of the instant methods. Similarly, although applicator nozzles are not required, when present, appropriate applicator nozzles include removable nozzles, disposable nozzles, and nozzles that are non-removable and/or non-disposable.

Regardless of whether the foregoing or functionally related or differing devices are used, and regardless of whether used “wet” or “dry”, ultrasonic energy is delivered to patient tissue without direct contact between the transducer tip and/or applicator nozzle and the treated patient tissue.

In certain embodiments, the generator includes a treatment algorithm that calculates an approximate treatment time. Alternatively, a physician or health professional can select the desired treatment time. For example, treatment time may be determined based on the area of the tissue for which treatment is desired.

Generally, treatment times vary from approximately 1 minute to approximately 25 minutes. However, shorter (approximately 30 seconds) and longer (25-30 minutes or greater than approximately 30 minutes) treatment times are contemplated. In certain embodiments, the length of time of a treatment comprises delivering ultrasonic energy to patient tissue for at least about 1 consecutive minute, at least about 2 minutes, at least about 3 minutes, or at least about 5 minutes. In certain embodiments, the length of time of a treatment comprises delivering ultrasonic energy to patient tissue for approximately 1-2 minutes, approximately 1-5 minutes, approximately 2-6 minutes, approximately 3-8 minutes, or approximately 4-10 minutes. In certain embodiments, the length of time of a treatment comprises delivering ultrasonic energy to patient tissue for approximately 5-15 minutes, approximately 5-20 minutes, or approximately 5-25 minutes. Note that the foregoing treatment times are approximate times per treatment. Thus, when the method comprises multiple treatments, the total treatment time over the course of one or more days, weeks, or months will be the aggregate of the individual treatment times.

In certain embodiments, the method comprises a single treatment (e.g., delivering ultrasonic energy once for a period of at least about 1 minute). In certain embodiments, the method comprises multiple treatments delivered over the course of one or more days, weeks, and/or months. In certain embodiments, the method comprises at least two treatments per week for at least two weeks. Note that when the method comprises multiple treatments, each treatment may be of the same duration or of differing durations.

Once emitted energy, and fluid spray when applicable, emerge from the applicator, the operator can direct the energy to the selected treatment site. In one embodiment, the treatment site is treated by slowly moving the applicator head back and forth and/or up and down (at a non-contact distance) across the site. The spray pattern may be, for example, serpentine or substantially checkerboard in pattern. This delivery method has two advantages. First, this method helps insure that ultrasonic energy and liquid spray are delivered to the entire treatment site. Second, this method may help prevent operator fatigue that would likely result if the device was held in substantially the same place throughout the treatment. In one embodiment, the applicator is held such that the ultrasonic energy and liquid spray are delivered substantially normal to the surface of the treatment site. Additionally, the spray pattern may include moving the applicator in-and-out relative to the wound surface (e.g., varying the distance from the wound while maintaining a non-contact distance). Such a spray pattern helps ensure that a treatment site is treated completely and at an effective distance.

The above described delivery method (whether used “wet” or “dry”) has two advantages. First, this method helps insure that ultrasonic energy and liquid spray are delivered to the entire treatment site. Second, this method may help prevent operator fatigue that would likely result if the device was held in substantially the same place throughout the treatment. In one embodiment, the applicator is held such that the ultrasonic energy and liquid spray are delivered substantially normal to the surface of the treatment site.

In other embodiments, the operator directs the energy to the treatment site by holding the applicator in substantially the same place throughout the treatment. Such a method is particularly useful when the treatment site is small and/or the treatment time is short. As noted above, the forgoing discussion of exemplary spray patterns are equally applicable whether ultrasonic energy is delivered “wet” or “dry”.

In certain embodiments, treatment is limited to effected tissue (e.g., tissue evincing visible symptoms). In certain embodiments, overtly effected tissue, as well as surrounding tissue that does not display visible symptoms, are treated. Without being bound by theory, treating peri-effected tissue may help prevent occurrence of overt symptoms in neighboring tissue.

In one embodiment, the need for a human operator is eliminated. The transducer assembly is affixed to a robotic arm programmed to direct the emitted energy and liquid spray to the treatment site. The robotic arm can be programmed to hold the applicator in substantially the same place throughout treatment or to move the applicator (back and forth or in and out) relative to the treatment site.

As outlined above, in certain embodiments the emitted ultrasonic energy and fluid spray (when applicable) are applied to the treatment site for a treatment time proportional to the size of the treatment site. In one embodiment, the invention provides a treatment algorithm for selecting treatment time based on the size of the treatment site. The time for each treatment is selected based on the area of the treatment site.

The present invention provides methods for using ultrasonic energy to treat an inflammatory skin disorder. By way of further non-limiting example, commonly-owned U.S. Pat. No. 6,569,099, and application Ser. Nos. 60/878,621, 11/473,934, 10/409,272, 10/815,384, and 12/006,739 disclose ultrasonic systems and devices that can be used in the subject methods. The entire contents of each of the foregoing patents and patent application are incorporated herein by reference. Briefly, these patents and applications discloses devices, systems, and methods for delivering ultrasound energy, in the presence or absence of a liquid spray, via an applicator. The ultrasound energy and, when present the liquid spray, is delivered from a non-contact distance. Commonly-owned U.S. patent Application Ser. Nos. 60/878,621, 11/473,934 and 12/006,739, the entire contents of which are incorporated herein by reference, additionally provide several examples of removable applicator nozzles that can be used with an ultrasound therapy device. The disclosed devices and systems can be used to deliver ultrasonic energy.

### (iv) Inflammatory Skin Disorders

The present invention is based in part on the observation that low frequency ultrasound energy delivered from a non-contact distance decreases the inflammatory response. As such, the present invention can be used to treat or ameliorate one or more symptoms of an inflammatory skin disorder by, for example, decreasing the inflammatory response. Exemplary inflammatory skin disorders include, but are not limited to, acne, rosacea, psoriasis, atopic dermatitis, seborrheic dermatitis, and contact dermatitis. Further exemplary inflammatory skin disorders include, but are not limited to, boils, carbuncles, pemphigus, cellulitis, Grover's disease, hidradenitis suppurativa, and lichen planus.

Over the course of one or more treatments, observable therapeutic efficacy is observed. Even a single treatment is therapeutically efficacious, regardless of whether such results are directly observable or observable only with molecular analysis of the lesioned tissue. The invention contemplates the use of low frequency ultrasonic energy to decrease one or more symptoms (including both overtly observable symptoms and symptoms observable at the molecular level) of an inflammatory skin disorder. Exemplary symptoms include, but are but limited to, expression of one or more markers of the inflammatory response, bacterial count, swelling, redness, itchiness, pain, number of lesions, frequency of outbreaks of lesions, severity of outbreaks of lesions, skin dryness, skin flaking, skin discoloration, scarring, etc.

### Acne

Acne is an inflammatory skin disorder of the skin's sebaceous glands and hair follicles. Acne, of various levels of severity, is estimated to affect about 80% of people between the ages of 12 and 24. However, acne is not a condition exclusively of teenagers. Stress, hormonal shifts, diet, and other factors can lead to acne in adults. For example, hormonal shifts, whether due to adolescence, menstruation, or pregnancy, can lead to production of large quantities of sebum. Sebum is an irritant that can clog the pores and form pimples. Pimples can become infected and form pustules.

The inflammatory response is an important component of the immune system. However, the inflammatory response can destroy healthy tissue and cause tissue damage.

Acne is an inflammatory skin condition. The primary symptoms involve superficial skin breakouts (e.g., white heads, blackheads, and pustules). The skin breakouts may become infected with acne bacteria (typically Propionibacteria acnes) that live in the hair follicles, potentially exacerbating the severity of the skin disorder.

To briefly summarize the inflammatory reaction that typically occurs in acne, in the acne lesions, transcription factors important for up-regulation of inflammatory cytokines (e.g., NF-KB) are activated. This results in up-regulation of inflammatory mediators, such as TNF-α and IL-1β. These cytokines act to help stimulate proliferation of secondary cytokines, such as IL-8, and also trigger the activation of MAP kinases (mitogen-activated protein kinase).

The present invention decreases the inflammatory response, thereby decreasing or lessening the skin irritations experienced with acne. Therapeutic efficacy can be assessed based on, for example, decrease in the number and/or severity of acne break outs over time (e.g., over the course of treatment).

Severe acne may result in significant scarring that can cause disfigurement, even after the acne lesions have subsided. By decreasing the underlying inflammatory response, as well as the symptoms of acne, the invention can also help decrease or eliminate acne-related scarring.

Acne is a multifactorial disorder related to the formation of comedones, hormonal stimulation, bacterial colonization, and host inflammatory response. Five major factors are involved in the pathophysiology of acne: abnormal keratinization of the hair follicle, increased androgens, excess sebum production, Propionibacterium acnes, and host immune response (e.g., the inflammatory cascade initiated by the irritation of the hair follicle and/or the increased presence of P. acnes).

P. acnes is an anaerobic aero-tolerant lipophilic diptheroid. It is typically found in the hair follicle, even of healthy skin, and thrives in triglyceride-rich sebum. P. acnes is a potent inflammatory stimulus, activating complement by both the classical (involving antibodies) and alternative pathways.

Acne vulgaris primarily occurs on the face and, to a lesser extent, on the torso including the back, chest, and shoulders. Most acne patients have increased sebum production which presents as oily skin. Acne lesions include closed comedones (whiteheads) and open comedones (blackheads). Inflammatory lesions can be superficial erythematous papules and pustules or deep-seated pustules and “cystic” nodules. Inflammatory lesions often heal with residual erythema or pigmentary change that may persist for months after the initial acne lesions have cleared. Permanent scarring can occur, especially with larger inflammatory lesions or severe acne that persists for long periods of time. Scarring is exacerbated when patients scratch, pick, or squeeze the lesions.

Acne is classified into five grades, based on the predominant type of lesion:
Grade 1—primarily comedonal
Grade 2—inflammatory papules and pustules
Grade 3—nodules/cysts
Grade 4—severe scarring
Grade 5—acne conglobata with sinus tracts

The methods of the present invention can be used in the treatment of acne of any grade. Depending on the severity of the acne symptoms, more treatments and/or longer treatment times (time of each treatment) may be needed to produce the desired therapeutic efficacy. Note, however, although dramatic improvement in patient appearance may take multiple treatments, even a single treatment delivers therapeutically effective doses of energy that penetrate the skin and begin to act on patient tissue. Overtime, the therapeutic efficacy of the individual treatments is additive or even synergistic, thus resulting in a decrease or elimination of symptoms and/or a lessening in the frequency of symptoms.

The present invention provides methods for treating acne in a patient in need thereof. By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response. Exemplary symptoms include, but are not limited to, expression of one or more markers of the inflammatory response, bacterial count, swelling, redness, itchiness, pain, number of lesions, frequency of outbreaks of lesions, severity of outbreaks of lesions, skin dryness, skin flaking, and skin discoloration.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Rosacea

Rosacea is an inflammatory skin disorder sometimes confused with adult acne. It begins as erythema (flushing and redness) on the central face and across the cheeks, nose, or forehead but can also less commonly affect the neck and chest. As rosacea progresses, other symptoms can develop such as semi-permanent erythema, telangiectasia (dilation of superficial blood vessels on the face), red bumps and pustules, red gritty eyes, burning and stinging sensations, and in some advanced cases, rhinophyma.

There are four identified rosacea subtypes and patients may have symptoms characteristic of more than one subtype.

1. Erythematotelangiectatic rosacea: This subtype is characterized by persistent redness (erythema) with a tendency to flush and blush easily. Telangiectasis is also a common symptom. Some patients report burning or itching sensations.

2. Papulopustular rosacea: This subtype is characterized by persistent redness with papules and some pus filled pustules.

3. Phymatous rosacea: This subtype is most commonly associated with rhinophyma. Symptoms also include thickening skin, irregular surface nodularities, and enlargement, primarily of the nose, chin (gnatophyma), forehead (metophyma), cheeks, eyelids (blepharophyma), and ears (otophyma). Telangiectasis may also be present.

4. Ocular rosacea: The most common symptoms of this subtype are red, dry and irritated eyes and eyelids.

Current treatments for rosacea include retinoids and antibiotics. However, these treatments are generally not suitable for long term therapy. Accordingly, the delivery of low frequency ultrasound, as described herein, offers a safe and effective treatment for rosacea.

The methods of the present invention can be used in the treatment of rosacea of any subtype. Depending on the severity of the symptoms, more treatments and/or longer treatment times (time of each treatment) may be needed to produce the desired therapeutic efficacy. Note, however, although dramatic improvement in patient appearance may take multiple treatments, even a single treatment delivers therapeutically effective doses of energy that penetrate the skin and begin to act on patient tissue. Over time, the therapeutic efficacy of the individual treatments are additive or even synergistic, thus resulting in a decrease or elimination of symptoms and/or a lessening in the frequency of symptoms. Over the course of one or more treatments, low frequency ultrasound can be used to decrease or eliminate symptoms of rosacea. In certain embodiments, the subject methods decrease the frequency and/or severity of rosacea outbreaks. Similarly, the present methods can help prevent scarring associated with rosacea.

The present invention provides methods for treating rosacea (of any subtype) in a patient in need thereof. By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response. Exemplary symptoms include, but are not limited to, expression of one or more markers of the inflammatory response, bacterial count, swelling, redness, itchiness, pain, number of lesions, frequency of outbreaks of lesions, severity of outbreaks of lesions, skin dryness, skin flaking, and skin discoloration.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Psoriasis

Psoriasis is an inflammatory skin condition caused, at least in part, by an inflammatory response in the patient. There are several major types, each with unique signs and symptoms. Between 10% and 30% of people who develop psoriasis get a related form of arthritis called “psoriatic arthritis,” which causes inflammation of the joints.

Plaque psoriasis is the most common type of psoriasis. About 80% of people who develop psoriasis have plaque psoriasis, which appears as patches of raised, reddish skin covered by silvery-white scales. These patches, or plaques, frequently form on the elbows, knees, lower back, and scalp. However, the plaques can occur anywhere on the body. Aside from the self-consciousness and cosmetic impact of these plaques, they are also itchy and uncomfortable. At times, they may bleed and become even more noticeable. The present invention provides novel methods for treating the symptoms of psoriasis. Specifically, low frequency ultrasound can be used to decrease the inflamed, scaly lesions associated with psoriasis. Additionally, low frequency ultrasound can help decrease the frequency of outbreaks.

Psoriasis is typically characterized as follows:

Plaque psoriasis (psoriasis vulgaris) is the most common form of psoriasis, accounting for 80-90% of psoriasis cases. Plaque psoriasis typically appears as raised areas of inflamed skin covered with silvery white scaly skin.

Flexural psoriasis (inverse psoriasis) appears as smooth inflamed patches of skin. It typically occurs in skin folds, such as around the genitals, armpits, or under the breasts.

Guttate psoriasis is characterized by numerous small oval (teardrop-shaped) spots. These numerous spots of psoriasis appear over large areas of the body, such as the trunk, limbs, and scalp. This type of psoriasis is associated with streptococcal throat infection, further supporting the link between psoriasis and the immune response.

Pustular psoriasis appears as raised bumps that are filled with non-infectious pus (pustules). The skin under and surrounding pustules is red and tender. Pustular psoriasis can be localized, generally to the hands and feet, or it can occur as patches occurring randomly on any part of the body.

Nail psoriasis produces changes in the appearance of finger and toe nails. Symptoms include discoloration, pitting, thickening of the skin under the nail, loosening of the nails, and crumbling of the nails.

Psoriatic arthritis involves joint and connective tissue inflammation, generally the joints of the fingers and toes. About 10-15% of people who have psoriasis also have psoriatic arthritis. Ultrasound therapy is especially well suited to treating psoriatic arthritis because the energy can readily penetrate to reach the joints of the fingers and toes.

Erythrodermic psoriasis involves the widespread inflammation and exfoliation of the skin over most of the body surface. It may be accompanied by severe itching, swelling and pain. It is often the result of an exacerbation of unstable plaque psoriasis, particularly following withdrawal of systemic treatment. This form of psoriasis can be fatal, as the extreme inflammation and exfoliation disrupts thermo-regulation and the barrier function of the skin.

Psoriasis has a significant impact on the quality of life of its suffers. Not only is the condition uncomfortable, and sometimes even painful, the associated skin lesions affect patient self-esteem. Current therapies include powerful immunosuppressants, retinoids, antibiotics, and cell cycle inhibitors. These therapies can have significant side effects. Accordingly, there is a need for improved methods of treating the symptoms of psoriasis. One or more treatments with low frequency ultrasound can be used to treat or ameliorate the symptoms of psoriasis, and can also be used to decrease the frequency of outbreaks.

The methods of the present invention can be used in the treatment of psoriasis of any subtype. Depending on the severity of the symptoms, more treatments and/or longer treatment times (time of each treatment) may be needed to produce the desired therapeutic efficacy. Note, however, although dramatic improvement in patient appearance may take multiple treatments, even a single treatment delivers therapeutically effective doses of energy that penetrate the skin and begin to act on patient tissue. Overtime, the therapeutic efficacy of the individual treatments are additive or even synergistic, thus resulting in a decrease or elimination of symptoms and/or a lessening in the frequency of symptoms. In the case of psoriasis, this includes not only the skin-related symptoms, but also the arthritis symptoms experienced by some sufferers.

The present invention provides methods for treating psoriasis (of any subtype) in a patient in need thereof. By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response. Exemplary symptoms include, but are not limited to, expression of one or more markers of the inflammatory response, bacterial count, swelling, redness, itchiness, pain, number of lesions, frequency of outbreaks of lesions, severity of outbreaks of lesions, skin dryness, skin flaking, and skin discoloration.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Atopic Dermatitis

Atopic dermatitis is a recurrent, inflammatory condition often experienced by infants, children, and young adults. It begins on the cheeks and may extend to the rest of the face, neck, wrists, and hands. The most common symptoms include intense itching and very dry skin.

Current treatments include the use of moisturizing creams and oils intended to combat the dry skin and itching associated with atopic dermatitis. Hydro-cortisone creams (1% or 2.5%) and tar preparations may also be used.

Atopic dermatitis is also known as atopic eczema. The skin of patients with this condition is especially sensitive to irritants and other allergens. The patient is thus vulnerable to skin reactions that cause red, dry, itchy skin. The itching often makes patients scratch or rub the effected tissue, and this can cause bleeding, cracking, oozing, or otherwise disrupt the skin. The open areas of skin can leave patients vulnerable to infection.

The present invention provides methods for treating atopic dermatitis in a patient in need thereof. By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response. Exemplary symptoms include, but are not limited to, expression of one or more markers of the inflammatory response, swelling, redness, itchiness, pain, number of lesions, frequency of outbreaks of lesions, severity of outbreaks of lesions, skin dryness, skin flaking, and skin discoloration.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Seborrheic Dermatitis

Seborrheic dermatitis is a chronic inflammatory skin disorder that is usually confined to areas of the head and trunk where sebaceous glands are prominent. Symptoms often include dull yellowish and/or reddish lesions, scaliness, and itching.

Current treatment primarily aims to decrease sebum or remove excess sebum from the effected area, thereby decreasing the presence of the irritating agent. Other treatments include the use of antifungal preparations (selenium sulfide, pyrithione zinc, azole agents, and topical terbinafine) and anti-inflammatory agents (topical steroids).

The present invention provides methods for treating seborrheic dermatitis in a patient in need thereof. By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response. Exemplary symptoms include, but are not limited to, expression of one or more markers of the inflammatory response, swelling, redness, itchiness, pain, number of lesions, frequency of outbreaks of lesions, severity of outbreaks of lesions, skin dryness, skin flaking, and skin discoloration.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Contact Dermatitis

Another form of dermatitis is contact dermatitis, which manifests itself as a rash that results from skin contact with an allergen or irritant. The primary symptom is itching. The itching can become so severe as to disturb sleep and normal activities. Additionally, scratching or rubbing of the irritated skin can lead to further skin damage and inflammation, including oozing or bleeding tissue.

Treatment for contact dermatitis includes avoiding contact with the irritating agent, if known. Additional treatment also includes applying cool compresses, calamine lotion, or hydrocortisone creams.

The present invention provides methods for treating contact dermatitis (regardless of the particular triggering agent) in a patient in need thereof. By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response. Exemplary symptoms include, but are not limited to, expression of one or more markers of the inflammatory response, swelling, redness, itchiness, pain, number of lesions, frequency of outbreaks of lesions, severity of outbreaks of lesions, skin dryness, skin flaking, and skin discoloration.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the uneffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Boils and Carbuncles

A boil, also referred to as a skin abscess, is a localized infection deep in the skin. A boil generally starts as a reddened, tender area. Over time, the area becomes firm and hard. Eventually, the center of the abscess softens and becomes filled with pus. Finally, the pus forms a “head,” which can be surgically opened or allowed to spontaneously drain out through the surface of the skin. Carbuncles are a particular type of boil typically caused by the bacterium Staphylococcus aureus. A sub-type of carbuncles, referred to as furuncles, usually have one or more opening onto the skin and are sometimes accompanied by fever or chills.

There are numerous causes for boils. For example, some boils are caused by ingrown hairs, or by a splinter or other foreign body that becomes lodged in the skin and causes a local irritation or infection. Regardless of the cause, treatments that decrease the inflammation and pain associated with the boil are useful for decreasing patient discomfort. Additionally or alternatively, treatments that address the underlying infection are useful for promoting healing of the abscess.

Although anyone can develop a boil, individuals who are immunosuppressed may be more susceptible to boils. Further, healing of the boil may be slower in individuals who are in poor health or are immunosuppressed. By way of example, boils may be more likely to develop and/or heal more slowly in diabetics, individuals with kidney failure, and individuals who are immunosuppressed due to a disease or medication. Examples of diseases that suppress the immune system and may increase the likelihood that boils develop include, but are not limited to, hypogammaglobulinemia and HIV/AIDS infection. Examples of medications or treatments that suppress the immune system and may increase the likelihood that boils develop include, but are not limited to, prednisone, methotrexate, and chemotherapeutic agents.

Current treatment for boils include hot compresses, soaking the effected area (optionally in a bath containing epsom salts), and lancing/drainage of the boil. Sometimes topical or systemic antibiotics are also used to address any accompanying infection. Additionally, to help decrease the recurrence of boils, current treatments include prophylactic cleansing of the skin with pH-balanced skin cleansers to help decrease the likelihood of recurrent local irritation or infection.

The present invention provides methods for treating boils (including carbuncles) in a patient in need thereof. Ultrasonic energy is administered over the course of one or more treatments to patients in need thereof to decrease one or more symptoms of boils. By way of non-limiting example, ultrasonic energy can be administered over the course of one or more treatments to decrease one or more of lesion size, number of lesions, severity of the lesions, frequency of recurrence of the lesions, itching, redness, pain associated with the lesions, bacteria count in the lesions, and swelling.

By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the unaffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Pemphigus

Pemphigus is a non-contagious, autoimmune disease of the skin and mucous membranes that causes vesicles (blisters), bullae and raw sores. In pemphigus, autoantibodies form against desmoglein. When autoantibodies attack desmogleins, the cells become separated from each other and the epidermis becomes “unglued”, a phenomenon referred to as acantholysis. This causes blisters that slough off and turn into sores. In some cases, these blisters may cover a significant area of the skin.

Two categories of pemphigus are pemphigus vulgaris (PV) and pemphigus foliaceus (PF). Pemphigus vulgaris is the most commonly diagnosed form of the disease. The sores and blisters are painful and delicate. For example, sometimes just touching the skin can cause it to tear. PV does not cause permanent scaring unless there is infection associated with the sore. With current therapies, the mortality rate is approximately 5% to 15%. Mortality is typically due to infection.

Pemphigus foliaceus is characterized by crusted sores or fragile blisters that typically first appear on the face and scalp. Over time, the sores occur on the chest and back. The blisters and sores are superficial and often itchy, but they are not typically as painful as the sores associated with PV. PF lesions can cause scarring. However, mortality from PF is lower than with PV.

Pemphigus is recognized by the appearance and distribution of the skin lesions. However, definitive diagnosis requires examination of a skin biopsy morphologically and for detection of anti-desmoglein autoantibodies.

If not treated, pemphigus can be fatal due to serious infection of the sores. The most common treatment is the administration of oral steroids, especially prednisone. The side effects of cortico-steroids may require the use of adjuvant drugs. The immuno-suppressant CellCept (mycophenolic acid) is among those being used. Intravenous gamma globulin (IV 1G) may be useful in severe cases. Mild cases sometimes respond to the application of topical steroids.

If skin lesions become infected, topical and/or systemic antibiotics may be prescribed. In addition, talcum powder is helpful to prevent oozing sores from adhering to bed sheets and clothes.

Other drugs that are sometimes used include: Dapsone®, gold injections, methotrexate, tetracycline, minocycline, or doxycycline combined with niacinamide. Further, the following biologics are currently in clinical trials: Rituxan, Remicade, and Enbrel.

The present invention provides methods for treating pemphigus in a patient in need thereof. Ultrasonic energy is administered over the course of one or more treatments to patients in need thereof to decrease one or more symptoms of pemphigus. By way of non-limiting example, ultrasonic energy can be administered over the course of one or more treatments to decrease one or more of lesion size, number of lesions, severity of the lesions, frequency of recurrence of the lesions, itching, redness, pain associated with the lesions, bacteria count in the lesions, and swelling.

By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the unaffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Cellilitis

Cellulitis is an infection of the subcutaneous tissue of the skin. Cellulitis can be caused by normal skin flora or by exogenous bacteria, and often occurs where the skin has previously been broken (e.g., cracks in the skin, cuts, blisters, burns, insect bites, surgical wounds, or catheter insertion sites). The current primary treatment for cellulitis is with antibiotics to address the infection.

Group A Streptococcus and Staphylococcus are the most common bacteria associated with cellulitis. These bacteria are part of the normal flora of the skin. However, following a break in the skin, these bacteria can lead to an infection.

The elderly and those with weakened immune systems are especially vulnerable to contracting cellulitis. Diabetics are more susceptible to cellulitis than the general population. Immunosuppressive drugs, HIV, and other illnesses or infections that weaken the immune system are also factors that make cellulitis infection more likely and serious. Chickenpox and shingles often result in blisters which break, providing a gap in the skin through which bacteria can enter. Diseases that affect blood circulation in the legs and feet, such as chronic venous insufficiency and varicose veins, are also risk factors for cellulitis.

Current treatment consists of resting the affected limb or area and treatment with oral or intravenous antibiotics. Flucloxacillin monotherapy is often sufficient in mild cellulitis. However, in more serious cases, combination therapy that further includes one or more of phenoxymethylpenicillin, benzylpenicillin, or ampicillin/amoxicillin is typically recommended. Additionally, medications to relieve the associated pain are often prescribed. However, pain levels should be monitored closely. Excessive pain may be a symptom of more serious infection, including the possibility of necrotizing fasciitis which requires emergency surgical attention.

The present invention provides methods for treating cellulitis in a patient in need thereof. Ultrasonic energy is administered over the course of one or more treatments to patients in need thereof to decrease one or more symptoms of cellulitis. By way of non-limiting example, ultrasonic energy can be administered over the course of one or more treatments to decrease one or more of lesion size, number of lesions, severity of the lesions, frequency of recurrence of the lesions, itching, redness, pain associated with the lesions, bacteria count in the lesions, and swelling.

By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the unaffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Grover's Disease

Grover's disease (transient acantholytic dermatosis) is a condition that appears suddenly as itchy red spots on the trunk, most often in older men. Most cases of Grover's disease last six to twelve months. However, symptoms may persist for a longer period of time.

Current treatment for minor outbreaks includes the use of prescription strength topical cortisone creams. For more significant outbreaks, Accutane or tetracycline therapy for one to three months is often prescribed. If these fail or the outbreak is severe, phototherapy treatments, antifungal pills and cortisone injections are alternatives.

There is currently no cure for Grover's disease. Recommended therapeutic regimens that may help alleviate symptoms include one or more of the following:
Remain cool and avoid sweating
Apply diphemanil methylsulfate powder
Apply a mild topical steroid such as hydrocortisone formulated as a lotion
Apply a moisturizing creams and/or antipruritic lotion. Lotions containing menthol and/or camphor are often recommended.
Apply calcipotriol cream
Phototherapy
Oral retinoids (e.g., acitretin or isotretinoin) have been reported to be helpful for some patients.

The present invention provides methods for treating Grover's disease in a patient in need thereof. Ultrasonic energy is administered over the course of one or more treatments to patients in need thereof to decrease one or more symptoms of Grover's disease. By way of non-limiting example, ultrasonic energy can be administered over the course of one or more treatments to decrease one or more of lesion size, number of lesions, severity of the lesions, frequency of recurrence of the lesions, itching, redness, pain associated with the lesions, bacteria count in the lesions, and swelling.

By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the unaffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Hidradenitis Suppurativa

Hidradenitis suppurativa is a chronic skin inflammation marked by the presence of blackheads and one or more red, tender, bumpy lesions. The lesions often enlarge, break open and drain pus. Scarring may result.

Although the cause of hidradenitis suppurativa is not known, it is generally considered to be a severe form of acne referred to as acne inversa. It occurs deep in the skin around sebaceous glands and hair follicles. Symptoms most commonly occur in skin of the groin and armpits. However, they may also occur in areas where skin rubs together, such as the inner thighs, under the breasts or between the buttocks. The symptoms may be localized to one area or multiple areas of the body.

Signs and symptoms of hidradenitis suppurativa include:
Small pitted areas of skin containing blackheads
One or more red, tender bumpy lesions that fill with pus. Note that the bumps often enlarge, break open and drain pus that has an unpleasant odor.
Itching, burning and excessive sweating may accompany the bumpy lesions.
Painful, pea-sized lumps that grow under the skin. These hard lumps, which may persist for years, can enlarge and become inflamed.
Painful bumps or sores that continually leak fluid. These heal very slowly, if at all, often leading to scarring and the development of tunnels under the skin.

There is currently no cure for hidradenitis suppurativa. However, there are some current treatment that aim to manage the symptoms of the condition. For mild cases, current treatment includes the use of warm compresses and regular cleansing of the lesions. If symptoms are more severe, current treatments include the use of topical or oral medications. These medications include: antibiotics (to address infection), retinoids (to decrease oil production), non-steroidal anti-inflammatories (to decrease pain and swelling), and corticosteroids (to decrease inflammation). Additionally or alternatively, surgical intervention may be needed to drain and/or remove lesions. If the extent of surgery is significant, skin grafting may be required to promote complete closure of the area.

The present invention provides methods for treating hidradenitis suppurativa in a patient in need thereof. Ultrasonic energy is administered over the course of one or more treatments to patients in need thereof to decrease one or more symptoms of hidradenitis suppurativa. By way of non-limiting example, ultrasonic energy can be administered over the course of one or more treatments to decrease one or more of lesion size, number of lesions, severity of the lesions, frequency of recurrence of the lesions, itching, redness, pain associated with the lesions, bacteria count in the lesions, and swelling.

By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the unaffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

### Lichen Planus

Lichen planus is a common inflammatory disease of the skin and mouth affecting approximately 1-2% of the population. Although the exact cause is not know, dennatologists generally categorize it as an autoimmune disease.

Lichen planus of the skin is characterized by reddish-purple, flat-topped bumps that may be very itchy. Some lesions may have a white lacy appearance called Wickham's Striae. Lichen planus lesions may occur anywhere on the body, but are most commonly observed on the wrists and ankles, as well as on the lower back, neck, legs, and genitals. In rare cases, the lesions occur in the scalp and/or nails. The lesions don't usually blister, except for in cases of the condition referred to as bullous lichen planus.

Symptoms of many cases of lichen planus subside within two years. However, as the lesions heal, lichen planus often leaves a dark brown discoloration on the skin.

Currently, there is no known cure for lichen planus. Treatments focus on relieving itching, decreasing inflammation of the skin tissue, improving the appearance of affected tissue until the rash subsides, and preventing scarring. The two most common treatments include the use of topical corticosteroid creams, ointments, or other anti-inflammatory drugs; oral cortisone; ultraviolet light treatment (PUVA); and oral antihistamines.

The present invention provides methods for treating lichen planus in a patient in need thereof. Ultrasonic energy is administered over the course of one or more treatments to patients in need thereof to decrease one or more symptoms of lichen planus. By way of non-limiting example, ultrasonic energy can be administered over the course of one or more treatments to decrease one or more of lesion size, number of lesions, severity of the lesions, frequency of recurrence of the lesions, itching, redness, skin discoloration, pain associated with the lesions, bacteria count in the lesions, and swelling.

By “treating” is meant to include decreasing or eliminating symptoms of the inflammatory skin disorder. Low frequency ultrasound energy is administered (with or without a liquid spray) to effected tissue of the patient. The low frequency ultrasound energy is administered without contact between the effected tissue and the ultrasound transducer or other components of the device (non-contact distance). The low frequency ultrasound energy penetrates the tissue to provide a therapeutic effect. Over the course of one or more treatments, the ultrasound energy decreases or eliminates the presence, severity, and/or frequency of the symptoms of the inflammatory skin disorder by, for example, decreasing the local inflammatory response.

In certain embodiments, the method is used to decrease or eliminate scarring associated with the inflammatory skin disorder. In other words, low frequency ultrasonic energy is administered to decrease the presence or severity of scarring, or even to eliminate scarring associated with the inflammatory skin disorder.

In certain embodiments, the low frequency ultrasound energy is administered locally to effected tissue, but without substantial contact with non-effected tissue. In other embodiments, the low frequency ultrasound energy is administered locally throughout the effected area—including both the effected tissue and the unaffected tissue in the same area of the body.

In certain embodiments, the low frequency ultrasound energy is administered as part of a therapeutic regimen. In other words, patients are also treated with one or more additional therapeutic modalities. In other embodiments, the low frequency ultrasound therapy (in the presence or absence of liquid spray) is the only therapeutic administered, and patients do not also use other therapeutic modalities.

The above conditions are exemplary of the inflammatory skin disorders that can be treated using non-contact, low frequency ultrasound therapy. Any of the features of the methods described herein can be used in the treatment of any of the diseases or conditions described herein. In other words, low frequency ultrasound therapy (with or without the use of a liquid spray) can be used in the treatment of any inflammatory skin disorder, for example the inflammatory skin disorders described herein. Further, the invention contemplates the use of one or more treatments with ultrasonic energy (in the presence or absence of liquid spray; alone or as part of a therapeutic regimen combined with one or more additional treatment modalities) to decrease or ameliorate one or more symptoms of an inflammatory skin disorder and/or to decrease expression of TNFα in symptomatic skin and/or tissue.

### (v) Combination Therapy

In certain embodiments, the use of non-contact ultrasound (with or without liquid spray) is used to treat an inflammatory skin disorder (e.g., to decrease the symptoms of the inflammatory skin disorder) and is used to decrease or avoid the need for using traditional topical, injectable, or oral agents. For example, by using non-contact ultrasound therapy, the need for treatment using steroids, antibiotics, anti-inflammatories, astringents, etc., can be decreased or eliminated.

In other embodiments, however, non-contact ultrasound therapy is used in combination with other treatment modalities as part of a therapeutic regimen for treating an inflammatory skin disorder. When used in this way, non-contact ultrasound therapy can act additively or synergistically with other treatments. Exemplary therapies include, but are not limited to, antibiotics, hydrocortisone creams, benzoil peroxide, retinoids and other vitamin A based agents, steroids or other immunosuppressive agents (methotrexate, cyclosporin), and the like. Further exemplary therapies include cytokine antagonists, such as TNF-α antagonists designed to decrease expression of TNF-α. Further exemplary therapies include phototherapy, a specialized dietary regimen, acupuncture, stress management, exercise, and the like.

Some of the available therapies for inflammatory skin disorders have significant side-effects. One potential benefit of ultrasound therapy is the avoidance of administering drugs such as steroids, systemic anti-inflammatory agents, or immunosuppressive agents. However, for certain conditions or patients, drug therapy may still be needed to provide additional therapeutic benefit and, when needed, can be used in combination with ultrasound therapy. In certain embodiments, ultrasound therapy decreases the patient's dependence on drug therapy. In other words, the additive or synergistic effects of ultrasound energy and the administered medicament allow the achievement of the same or better therapeutic efficacy at a decreased dose of drug.

When drug therapy is used in combination with ultrasound, the invention contemplates that the drug itself may be delivered via the ultrasound device. In other words, the liquid drug is delivered to an ultrasound transducer to generate a liquid spray, and the liquid spray and ultrasound energy are delivered to the patient and penetrate the patient's tissue. The invention also contemplates that the medicament can be topically applied directly to the patient tissue, and ultrasound energy (with or without an inert or medicated liquid spray) can be delivered to the topically applied medicament and to the underlying patient tissue. When used in this manner, the ultrasound energy facilitates the penetration of the drug into the patient's tissue. Additionally, the invention contemplates embodiments in which the medicament is administered as per its labeling instructions (e.g., topically, orally, intravenously, etc.) and ultrasound energy.

In other embodiments, the drug is delivered as part of a therapeutic regimen, but ultrasound energy is not used to facilitate delivery.

Regardless of the mechanism by which drug is delivered, the invention contemplates combinatorial therapies that involve administering a drug as part of the therapeutic regimen. Exemplary drugs include, but are not limited to, corticosteroids, analgesics, non-steroidal anti-inflammatory agents, vitamin A derivatives, vitamin D derivatives, anti-mitotic agents, TNF-α inhibitory agents, antibiotics, anti-fungals, and immunosuppressants.

### (vi) Animal Models

The use of low frequency ultrasonic energy (in the presence or absence of liquid spray; in the presence or absence of additional therapeutic modalities) to treat one or more symptoms of an inflammatory skin disorder can be tested in one or more animal models. Exemplary animal models are described briefly herein. However, numerous animal models exist and any model available in the art can be readily used to evaluate a particular treatment regimen (e.g., to evaluate number of treatments, duration of treatment, combination with one or more current treatment modalities, etc).

Numerous models for dermatitis exist and can be used. The keratin 14 IL-4 transgenic mouse model and the WBN/Kob-Ht rat model are commonly used. See, for example, Chen et al. (2005) Clin Exp Immunolo 142: 21-30 and Asakawa et al. (2005) Exp Animals 54: 461-465. Additional animal models are summarized in Nishimuta and Ito (2003) Archives of Dermatol Res 294: 544-551.

Animal models for acne include UV light-induced lesions in guinea pigs and the croton oil-induced cutaneous inflammation model. Acne is also studied using edema models. For example, edema is induced in the ear of a rodent by injecting arachidonic acid or carrageenan. Efficacy of a treatment regimen in assessed by evaluating reduction in agent-induced edema relative to a control. Additional animal models include the use of rodents injected subcutaneously with heat killed P. acnes bacterium. Further models are summarized in Nishimuta and Ito (2003) Archives of Dermatol Res 294: 544-551.

Animal models for cellulitis include mouse models summarized by Bisno and Gaviria (1997) Infection and Immunity 65: 4926-4930. Briefly, cellulitis symptoms are induced in mice by injecting animals with a strain of Group G streptococcus (strain 1750) originally isolated from the lesions of a patient with cellulitis. Efficacy of a treatment regimen is assessed in such mice relative to no treatment or a control treatment. Another exemplary animal model is an avian model in which cellulitis-like lesions are induced by subcutaneous injection of E. coli bacteria originally isolated from a cellulitis lesion.

Numerous animal models of psoriasis exist. 17 rodent models are summarized by Schon. See, Schon (1999) Society of Investigative Dermatology 112: 405-410. These animal models include spontaneous mutant models, transgenic animals, knock-out animals, and xenotransplantation models.

Rodent and canine models for pemphigus exist and are used. See, for example, Liu et al. (2004) Society for Investigative Dermatology DOI: 10.1111/j.0022-202X.2004.22438.x; Hurvitz (1980) American Journal of Pathology 98: 861-864. Additionally, pemphigus is often studied in organ culture using samples of lesioned tissue from affected individuals. See, Liu et al. Note that the use of organ culture to model and study inflammatory skin disorders can be used to study any of the conditions described herein. Briefly, samples of lesioned and healthy tissue are taken from an affected individual. These tissues are cultured in vitro.

Additional animal models (including mouse, rat, porcine, and canine models) of various inflammatory skin disorders, as well as more detailed descriptions of many of the foregoing models, are provided in “Animal Models of Human Inflammatory Skin Diseases”, Lawrence S. Chan; published by Informa Health Care, Dec. 29, 2003.

Therapeutic regimens including one or more treatments with low frequency ultrasonic energy (alone or in combination with one or more additional treatment modalities) can be tested in one or more animal models. Exemplary models are described herein, although numerous additional animal models are well known in the art and can be similarly used. Treatment with ultrasonic energy is compared to, for example, no treatment controls or control treatment with one or more current, non-ultrasonic therapies. Additionally or alternatively, such models can be used to assess, for example, the effectiveness of a therapeutic regimen in which the frequency of treatments and/or the duration of each treatment are varied. Similarly, such models can be used to assess, for example, the effectiveness of a therapeutic regimen in which ultrasonic energy is delivered in the presence versus the absence of a fluid spray, as well as in the presence or the absence of a medicament (e.g., a fluid spray containing medicament and/or a topically applied medicament).

Additionally, therapeutic regimens including one or more treatments with low frequency ultrasonic energy (alone or in combination with one or more additional treatment modalities) can be tested in in vitro models (e.g., cell-based models, organ culture models). Further, such therapeutic regimens can be tested in vivo in human patients.

### (vii) Diagnostic Methods

One aspect of the present invention is based on the recognition that low frequency ultrasound can be used to decrease the inflammatory response by decreasing expression and/or activation of pro-inflammatory cytokines. As such, one aspect of the invention provides methods for decreasing expression and/or activation of one or more pro-inflammatory cytokines. The method can be performed on cells or tissue explants cultured or otherwise maintained in vitro. In such in vitro embodiments, cells or tissue explants in culture are contacted with low frequency ultrasound energy from a non-contact distance, as described throughout the application. The cells or tissue explants can be assessed to evaluate the decrease in expression and/or activation of one or more pro-inflammatory cytokines in comparison to untreated control. Exemplary pro-inflammatory cytokines that can be evaluated include, but are not limited to, TNFα, IL-1β, IL-8, p38 MAPK, other pro-inflammatory interleukins, and the like.

Suitable diagnostics methods can also be performed following in vivo treatment of tissues. Note that in this context the terms “in vitro” and “in vivo” are used to characterize the cells at the time of receiving the ultrasound treatment. Following treatment, the cells can be evaluated either in the context of the patient or animal or using an in vitro assay. The post-treatment evaluation method does not alter whether the ultrasound delivery occurred in vivo or in vitro.

In certain embodiments, ultrasound energy is delivered to effected tissue of a patient in need thereof (delivered in vivo), and expression and/or activation of one or more pro-inflammatory cytokines is evaluated following treatment. Expression and/or activation of one or more pro-inflammatory-cytokines can be evaluated at any one or more time points following one or more treatments, and compared to expression and/or activation prior to initiation of treatment (but after the onset of symptoms of the inflammatory disorder). When used in this way, decrease in the local inflammatory response, as assessed by expression and/or activation of one or more pro-inflammatory cytokines, can be used to evaluate the progress of the treatment.

As indicated above, although the ultrasound energy is delivered in vivo, analysis of the one or more pro-inflammatory cytokines can be conducted in vivo or in vitro. For example, for in vitro analysis, suitable tissue samples can be taken over time and analyzed in vitro. In the case of an inflammatory skin disorder, small skin samples can be taken for analysis. For in vivo analysis, vital dyes and agents can be used to help assess the inflammatory response in the tissue in its in vivo context and without the need to obtain a sample or biopsy from the patient. Regardless of whether the diagnostic step is conducted in vitro or in vivo, exemplary pro-inflammatory cytokines that can be evaluated include, but are not limited to, TNFα, IL-1β, IL-8, p38, other pro-inflammatory interleukins, and the like.

In certain embodiments, the diagnostic step is conducted multiple times throughout the course of treatment. In certain embodiments, the one or more diagnostic steps are used by a health care provider to help determine the duration of treatment, as well as whether ultrasound therapy should be used alone or combined with other therapies.

### Exemplifications

The invention now being generally described, it will be more readily understood by reference to the following examples, which are included merely for purposes of illustration of certain aspects and embodiments of the present invention, and are not intended to limit the invention.

### Example 1

### Low Frequency Ultrasound does not Decrease Viability of a Human Monocyte-Derived Cell Line Cultured In Vitro

A human monocyte-derived cell line (THP-1) was used to evaluate the ability of low frequency ultrasound to modulate an inflammatory response. Before evaluating whether low frequency ultrasound decreased an inflammatory response, we assessed whether the ultrasound energy altered cell viability.

THP-1 cells were cultured in RPMI 1640 medium supplemented with fetal calf serum (FCS; 10% v/v), L-glutamine (2 mM), penicillin (100 U/ml), streptomycin (100 ug/ml), and sodium pyruvate (1 mM). The cells were maintained at 37° C. and 5% CO₂.

2-4 ml of THP-1 cell suspension was prepared and exposed to 3 minutes of either low frequency ultrasound delivered by the MIST™ Therapy system (Mist; manufactured by Celleration, Inc.; www.celleration.com), or to kinetic energy delivered by a nebulizer (KE). A third sample was not exposed to any treatment (untreated). Note that the cells exposed to low frequency ultrasound were treated at a non-contact distance from the cells and from their culture medium. In other words, the low frequency ultrasound was delivered at a non-contact distance from the cells and their culture medium. Additionally, in this example, low frequency ultrasound energy was delivered alone—without simultaneous delivery of a spray or coupling medium (e.g., “dry”). However, the energy could have been delivered via a fluid spray (e.g., saline solution, water, etc.).

Following treatment, the viability of the THP-1 cells was assessed by determining the percentage of live cells in the sample following treatment. As depicted in FIG. 1, we consistently observed greater than 90% cell survival in all three groups (untreated group=left most bar; ultrasound energy group (Mist)=center bar; kinetic energy group (KE)=right most bar). Thus, viability of these cells was not significantly affected by treatment with low frequency ultrasound.

To confirm that any negative effect on cell viability was not delayed, the number and viability of treated THP-1 cells was assessed both immediately after treatment, and following an additional 18 hours of post-treatment culture. The total number and viability of the cells did not differ, thus supporting the conclusion that the ultrasound therapy treatment had no immediate or delayed cytotoxic effects on cell viability.

### Example 2

### Low Frequency Ultrasound Specifically Inhibited an Inflammatory Response

TNFα is a proinflammatory cytokine that is upregulated as part of the inflammatory response. We evaluated whether low frequency ultrasound could reduce the inflammatory response by assessing TNFα production in response to LPS stimulation.

THP-1 cell suspensions were prepared and exposed to 3 minutes of either low frequency ultrasound delivered by the MIST™ Therapy system, or to kinetic energy delivered by a nebulizer. A third sample was not exposed to any treatment. Note that the cells exposed to low frequency ultrasound were treated at a non-contact distance from the cells and from their culture medium. In other words, the low frequency ultrasound was delivered at a non-contact distance from the cells and their culture medium. Additionally, in this example, low frequency ultrasound energy was delivered alone—without simultaneous delivery of a spray or coupling medium (“dry”). However, the energy could have been delivered via a fluid spray (e.g., saline solution, water, etc.).

Following treatment, each group of cells was cultured in 96 well-plates and stimulated with LPS (LPS from E. coli, Sigma-Aldrich) to induce an inflammatory reaction. Cells were plated at concentrations of 1×10⁶ cells/ml or 0.5×10⁶ cells/ml. Cells were stimulated with either 10 or 100 ng/ml LPS. Following 5 hours of LPS stimulation, TNFα protein concentration in the cell culture supernatants was determined using a commercially available ELISA kit (R&D Systems).

The cells treated with low frequency ultrasound produced less TNFα following LPS stimulation than the untreated cells or the cells exposed to kinetic energy. As depicted in FIG. 2, low frequency ultrasound treatment inhibited TNFα production by LPS stimulated THP-1 cells in comparison to that of untreated cells or cells treated with kinetic energy. In FIG. 2, TNFα was measured by ELISA and the percentage of the OD of the treated cells versus the untreated cells is given on the y-axis (untreated group=left most bar; ultrasound energy group (Mist)=center bar; kinetic energy group (KE)=right most bar). Thus, low frequency ultrasound treatment inhibited the inflammatory response in these cells.

To confirm that delivery of ultrasound energy in combination with a liquid spray (“wet”) had a similar effect, the above experiment was repeated using the MIST system to deliver ultrasound energy and a saline spray. Briefly, 2-4 ml of THP-1 cell suspension was placed in a Petri dish and subjected to Mist ultrasound therapy with saline solution. The saline solution is delivered to the ultrasound transducer which produces an atomized spray of saline. Ultrasound energy and the spray of saline is thus delivered to the cells. Following the treatment time (in this experiment—3 minutes), the cell suspension was collected and processed, as described above for cells treated “dry”.

Treatment of the THP-1 cells with ultrasound energy and a saline spray (“wet”) had a similar effect on LPS-induced TNF-α production as the “dry” treatment. Both treatments resulted in inhibition of LPS-induced TNF-α production in comparison to untreated and KE (non-ultrasound) controls.

### Example 3

### Low Frequency Ultrasound Specifically Inhibited an Inflammatory Response

p38 is typically upregulated as part of the inflammatory response. We evaluated whether low frequency ultrasound could reduce the inflammatory response by assessing p38 activation following LPS stimulation. Activation of p38 is often evaluated by detecting p38 phosphorylation using, for example, an antibody that recognizes phosphorylated p38.

THP-1 cell suspensions were prepared and exposed to 3 minutes of either low frequency ultrasound delivered by the MIST™ Therapy system, or to kinetic energy delivered by a nebulizer. A third sample was not exposed to any treatment. Note that the cells exposed to low frequency ultrasound were treated at a non-contact distance from the cells and from their culture medium. In other words, the low frequency ultrasound was delivered at a non-contact distance from the cells and their culture medium. Additionally, in this example, low frequency ultrasound energy was delivered alone—without simultaneous delivery of a spray or coupling medium. However, the energy could have been delivered via a fluid spray (e.g., saline solution, water, etc.).

Following treatment, each group of cells was cultured in 6 well-plates and stimulated with LPS (LPS from E. coli, Sigma-Aldrich) to induce an inflammatory reaction. Cells were stimulated with either 100 ng/ml LPS (or with 0 ng LPS as an unstimulated control). Following 30 minutes of LPS stimulation, the cells were lysed in Laemmli buffer at a concentration of 20×10⁶ cell/ml. Cell lysates (10 ul) were separated electrophoretically, and analyzed by Western blot using an antibody to phospho-p38 MAPK (Thr180/Tyr182) primary antibody and an HRP-conjugated sheep anti-rabbit secondary antibody (Amersham). Protein detection was performed by chemiluminescence with ECL Plus Western Blotting Detection (Amersham).

Treatment with low frequency ultrasound inhibited p38 activation (decreased the amount of phosphorylated p38 detected in the assay). As depicted in FIGS. 3 and 4, low frequency ultrasound treatment inhibited p38 activation in LPS stimulated THP-1 cells in comparison to that of untreated cells or cells treated with kinetic energy. FIG. 3 shows Western blot analysis using anti-phosphorylated p38 antibody and FIG. 4 provides a quantitative analysis of the intensity of the band shown in FIG. 3. As can be seen from FIGS. 3 and 4, treatment with low frequency ultrasound inhibited p38 activation in LPS stimulated cells. Thus, low frequency ultrasound treatment inhibited the inflammatory response in these cells.

### Example 4

### Low Frequency Ultrasound Specifically Inhibited an Inflammatory Response

The experiment outlined above in Example 3 was repeated. However, in this experiment, activation of both p38 and hsp27 were evaluated by evaluating the phosphorylation of p38 and hsp27. Briefly, THP-1 cells were subjected to 3 min of MIST, control treatment, or were untreated. Phosphorylation of p38 and hsp27 in ±LPS treated cells was assessed by western blot analysis. As shown in FIG. 5, low frequency ultrasound treatment attenuated activation of p38 and hsp27.

### Example 5

### The Effect of Low Frequency Ultrasound on the Inflammatory Response was Dependent on Treatment Time

The period of exposure to the low frequency ultrasound (the treatment time) corresponds to the amount of energy delivered. As such, a longer treatment time results in delivery of more energy. We evaluated whether Mist treatment would have a larger effect on cells if used for a longer treatment time.

THP-1 cell suspensions were prepared and exposed to either 3 or 6 minutes of low frequency ultrasound delivered by the MIST™ Therapy system (“dry”). Control samples treated with kinetic energy delivered by a nebulizer (for 3 or 6 minutes) or left untreated were also evaluated. As before, the cells exposed to low frequency ultrasound were treated at a non-contact distance from the cells and from their culture medium. In other words, the low frequency ultrasound was delivered at a non-contact distance from the cells and their culture medium.

Following treatment, each group of cells was cultured in 96 well-plates and stimulated with LPS (LPS from E. coli, Sigma-Aldrich) to induce an inflammatory reaction. Cells were plated at concentrations of 1×10⁶ cells/ml or 0.5×10⁶ cells/ml. Cells were stimulated with either 10 or 100 ng/ml LPS. Following 5 hours of LPS stimulation, TNFα protein concentration in the cell culture supernatants was determined using a commercially available ELISA kit (R&D Systems).

This experiment indicated that the anti-inflammatory response was proportional to the length of treatment, across this range of treatment times. Specifically, TNF-α production in LPS stimulated cells was approximately 45% that of controls following 3 minutes of Mist treatment, but only approximately 25% that of controls following 6 minutes of Mist treatment. Thus, the larger amount of ultrasound energy delivered over the longer treatment time had a noticeable impact on TNF-α production.

### REFERENCES

Clark (1996). The Molecular and Cellular Biology of Wound Repair, New York, N.Y., Plenum, pages 3-50.
Janeway and Medzhitov (2002). Annual Review of Immunology 20: 197-216.
Dong et al. (2002) Annual Review of Immunology 20: 55-72.

### INCORPORATION BY REFERENCE

All publications and patents mentioned herein, are hereby incorporated by reference in their entirety as if each individual publication or patent was specifically and individually indicated to be incorporated by reference.

### EQUIVALENTS

Those skilled in the art will know or be able to ascertain using no more than routine experimentation, many equivalents to the embodiments and practices described herein. Accordingly, it will be understood that the foregoing descriptions are to be considered in all respects illustrative, rather than limiting, of the invention. For example, a variety of systems and/or methods may be implemented based on the disclosure and still fall within the scope of the invention. The specifications and other disclosures in the patents, patent applications, and other references cited herein are hereby incorporated by reference in their entirety. Further, the invention contemplates combinations of any of the foregoing aspects and embodiments of the invention.

We claim:

1. A method of treating an inflammatory skin disorder in a patient in need thereof, comprising delivering low frequency ultrasound energy from a non-contact distance to effected skin of the patient in need thereof, wherein said low frequency ultrasound energy penetrates the skin to provide a therapeutic effect to decrease one or more symptoms of the inflammatory skin disorder.

2. The method of claim 1, wherein the inflammatory skin disorder is acne.

3. The method of claim 1, wherein the inflammatory skin disorder is rosacea.

4. The method of claim 1, wherein the inflammatory skin disorder is psoriasis.

5. The method of claim 1, wherein the inflammatory skin disorder is atopic dermatitis, seborrheic dermatitis, or contact dermatitis.

6. The method of claim 1, wherein the inflammatory skin disorder is boils, carbuncles, pemphigus, or cellulitis.

7. The method of claim 1, wherein the inflammatory skin disorder is Grover's disease, hidradenitis suppurativa, or lichen planus.

8. The method of claim 1, wherein the low frequency ultrasound energy is delivered via a liquid spray, and the method comprises delivering the low frequency ultrasound energy and the liquid spray from a non-contact distance to effected skin of the patient in need thereof to provide a therapeutic effect.

9. The method of claim 1, wherein the low frequency ultrasound energy is delivered in the absence of a liquid spray or coupling agent.

10. The method of claim 1, wherein the low frequency ultrasound energy is approximately 30-50 kHz.

11. The method of claim 10, wherein the low frequency ultrasound energy is low intensity ultrasound energy.

12. The method of claim 1, wherein the method is part of a therapeutic regimen combining one or more additional treatment modalities.

13. A method for reducing scarring associated with an inflammatory skin disorder in a patient in need thereof, comprising
providing a transducer which can emit low frequency ultrasonic energy;
delivery said low frequency ultrasonic energy to effected skin of said patient;
wherein said ultrasonic energy is delivered from a non-contact distance between a vibrating tip of the transducer and said effected skin of said patient, and wherein the delivered ultrasonic energy provides a therapeutic effect to reduce scarring associated with said inflammatory skin disorder.

14. The method of claim 13, wherein the ultrasonic energy is delivered via a liquid spray, and the method comprises delivering the low frequency ultrasonic energy and the liquid spray to said patient.

15. The method of claim 14, wherein the liquid spray is generated by delivering liquid to a distal portion of the transducer.

16. The method of claim 13, wherein the ultrasonic energy is delivered in the absence of a liquid spray or coupling agent.

17. The method of claim 13, wherein the inflammatory skin disorder is selected from acne, rosacea, or psoriasis.

18. The method of claim 13, wherein the inflammatory skin disorder is selected from atopic dermatitis, seborrheic dermatitis, or contact dermatitis.

19. The method of claim 13, wherein the inflammatory skin disorder is selected from boils, carbuncles, pemphigus, cellulitis, Grover's disease, hidradenitis suppurativa, or lichen planus

20. A method for treating an inflammatory skin disorder, comprising
providing a transducer which can emit low frequency ultrasonic energy;
delivering said ultrasonic energy to a patient in need thereof;
wherein said ultrasonic energy is delivered from a non-contact distance between a vibrating tip of the transducer and treated patient tissue, and wherein the delivered ultrasonic energy provides a therapeutic effect to decrease one or more symptoms of said inflammatory skin disorder in said patient.

21. The method of claim 20, wherein the ultrasonic energy penetrates the patient tissue to provide a therapeutic effect.

22. The method of claim 20, wherein the ultrasonic energy is delivered via a liquid spray, and the method comprises delivering the low frequency ultrasonic energy and the liquid spray to the patient.

23. The method of claim 22, wherein the liquid spray is generated by delivering liquid to a distal portion of the transducer.

24. The method of claim 20, wherein the ultrasonic energy is delivered in the absence of a liquid spray or coupling agent.

25. The method of claim 20, wherein the method is part of a therapeutic regimen combining one or more additional treatment modalities.

26. The method of claim 20, wherein the one or more symptoms are selected from one or more of expression of an inflammatory cytokine, inflammation, pain, itching, skin dryness, skin flaking, bacterial count, number of skin lesions, severity of skin lesions, frequency of outbreaks of skin lesions, redness, and skin discoloration

27. The method of claim 20, wherein the inflammatory skin disorder is selected from acne, rosacea, or psoriasis.

28. The method of claim 20, wherein the inflammatory skin disorder is selected from atopic dermatitis, seborrheic dermatitis, or contact dermatitis.

29. The method of claim 20, wherein the inflammatory skin disorder is selected from boils, carbuncles, pemphigus, cellulitis, Grover's disease, hidradenitis suppurativa, or lichen planus

30. A method for managing symptoms of an inflammatory skin disorder, comprising
providing a transducer which can emit low frequency ultrasonic energy;
delivering said ultrasonic energy to a patient in need thereof for at least two consecutive minutes at least twice per week;
wherein said ultrasonic energy is delivered from a non-contact distance between a vibrating tip of the transducer and treated patient tissue, and wherein the delivered ultrasonic energy provides a therapeutic effect to treat one or more symptoms of said inflammatory skin disorder.

31. A method for decreasing the number of skin lesions on a patient suffering from an inflammatory skin disorder, comprising
providing a transducer which can emit low frequency ultrasonic energy;
delivering said ultrasonic energy to said patient;
wherein said ultrasonic energy is delivered from a non-contact distance between a vibrating tip of the transducer and treated patient tissue, and wherein the delivered ultrasonic energy provides a therapeutic effect to decrease the number of skin lesions on skin of said patient.

32. A method for decreasing expression of an inflammatory cytokine in a patient having an inflammatory skin disorder, comprising
providing a transducer which can emit low frequency ultrasonic energy;
delivering said ultrasonic energy to said patient in need thereof;
wherein the delivered ultrasonic energy decreases expression of said inflammatory cytokine in effected skin tissue of said patient.

33. The method of claim 32, wherein said inflammatory cytokine is TNFα.

34. The method of claim 32, wherein the ultrasonic energy is delivered via a liquid spray, and the method comprises delivering the low frequency ultrasonic energy and the liquid spray to the patient.

35. The method of claim 34, wherein the liquid spray is generated by delivering liquid to a distal portion of the transducer.

36. The method of claim 32, wherein the ultrasonic energy is delivered in the absence of a liquid spray or coupling agent.

37. The method of claim 1, wherein the ultrasonic energy is delivered at a frequency of approximately 20 kHz to 200 kHz.

38. The method of claim 1, wherein the ultrasonic energy is delivered at a frequency of approximately 30-50 kHz.

39. The method of claim 1, wherein the ultrasonic energy level provided to patient tissue is approximately 0.1-2.0 watts/cm².

40. The method of claim 1, wherein the ultrasonic energy is delivered with a liquid spray, and wherein the liquid spray is selected from a saline solution or other substantially inert liquid.

41. The method of claim 1, wherein the ultrasonic energy is delivered with a liquid spray, and wherein the liquid spray includes a therapeutic medicament.
